(19) United States
(12) Patent Application Publication
(10) Pub. No.: US 2026/0207642 A1
(43) Pub. Date: Jul. 23, 2026

(54) ORALLY-DELIVERED BETA-LACTAMASE INHIBITOR AND CEFTIBUTEN DOSING REGIMENS

(71) Applicant: Venatorx Pharmaceuticals, Inc., Malvern, PA (US)

(72) Inventors: Paul C. MCGOVERN, Berwyn, PA (US); Kathryn LOWE, Trappe, PA (US); Anthony DRAGER, Thorndale, PA (US)

(21) Appl. No.: 19/453,949

(22) Filed: Jan. 20, 2026

Related U.S. Application Data
(60) Provisional application No. 63/747,773, filed on Jan. 21, 2025.

(51) Int. Cl. A61K 31/69; A61K 9/00; A61K 9/48; A61K 31/546; A61P 31/04

(52) U.S. Cl. CPC A61K 31/69; A61K 9/0053; A61K 9/4825; A61K 9/485; A61K 9/4858; A61K 9/4866; A61K 31/546; A61P 31/04

(57) ABSTRACT

Disclosed herein is a method of treating a bacterial infection, the method comprising co-administering (i) a compound that is

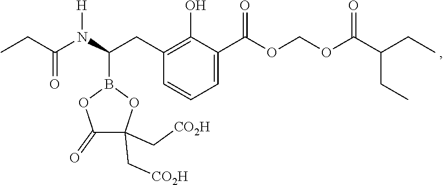 [SMILES: CCC(=O)N[C@@H](Cc1cccc(C(=O)OCOC(=O)C(CC)CC)c1O)B1OC(=O)C(CC(=O)O)(CC(=O)O)O1]

or a pharmaceutically acceptable salt or a solvate thereof; and (ii) ceftibuten in a daily amount greater than 400 mg.

### CROSS-REFERENCE

This application claims the benefit of U.S. Provisional Application Ser. No. 63/747,773 filed Jan. 21, 2025, which is hereby incorporated by reference in its entirety.

### STATEMENT AS TO FEDERALLY SPONSORED RESEARCH

This invention was made with government support under Contract number HHSN272201600029C awarded by the National Institutes of Health (NIH) and under Contract number 75A50123C00050 awarded by the Department of Health and Human Services (HHS), Administration for Strategic Preparedness and Response (ASPR), Biomedical Advanced Research and Development Authority (BARDA). The government has certain rights in the invention.

### BACKGROUND OF THE INVENTION

Antibiotics are the most effective drugs for treating bacterial infectious diseases. They are largely used in the clinic because of their good antibacterial effect with limited side effects. Among them, the beta-lactam class of antibiotics (for example, penicillins, cephalosporins, monobactams and carbapenems) are preferred because their effect is bactericidal, and their target is absent in eukaryotic cells with consequent low toxicity.

To counter the efficacy of the various beta-lactams, bacteria have evolved to produce variants of beta-lactam deactivating enzymes called beta-lactamases, and in the ability to share this tool both vertically and horizontally inter- and intra-species. These beta-lactamases are categorized as “serine” or “metallo” based, respectively, based on the presence of a key serine or zinc in the enzyme active site. The rapid induction, selection and spread of this mechanism of bacterial resistance can severely limit the whole class of beta-lactam treatment options in the hospital and in the community. There is a need for stable, effective, and safe therapeutic agents, combining a beta-lactam antibiotic and a beta-lactamase inhibitor, that can treat such resistant infections.

### SUMMARY OF THE INVENTION

Disclosed herein is a method of treating a bacterial infection in a subject in need thereof, the method comprising co-administering:
(i) a compound that is

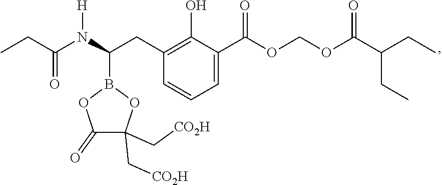 [SMILES: CCC(=O)N[C@@H](Cc1cccc(C(=O)OCOC(=O)C(CC)CC)c1O)B1OC(=O)C(CC(=O)O)(CC(=O)O)O1]

or a pharmaceutically acceptable salt or a solvate thereof, and
(ii) ceftibuten in a daily amount greater than 400 mg.

### BRIEF DESCRIPTION OF THE DRAWINGS

FIG. 1A shows comparative efficacy of the human-simulated exposures of ceftibuten (400 mg q8 h and 600 mg q12h) in combination with ledaborbactam (400 mg q8 h and 600 mg q12h, respectively) in the kidney (ceftibuten monotherapy assessment not included in studies with strains EC808 and KP1203).

FIG. 1B shows comparative efficacy of the human-simulated exposures of ceftibuten (400 mg q8 h and 600 mg q12h) in combination with ledaborbactam (400 mg q8 h and 600 mg q12h, respectively) in the bladder (ceftibuten monotherapy assessment not included in studies with strains EC808 and KP1203).

FIG. 2 shows the dissolution profile comparison of the fixed dose capsule prototype of ceftibuten dihydrate combined with Compound 1 citrate coordination complex (Compound A) and co-dosed capsules of ceftibuten dihydrate and the SEDDS formulation of Compound 1 ethanolate (Compound B).

### DETAILED DESCRIPTION

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

As used herein, the term “therapeutic” means an agent utilized to treat, combat, ameliorate, or improve an unwanted condition or disease of a patient.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted.

“Administering” a pharmaceutical composition may be accomplished by injection, topical administration, and oral administration or by other methods alone or in combination with other known techniques.

By “pharmaceutically acceptable,” it is meant the carrier, diluent or excipient must be compatible with the other ingredients of the composition and not deleterious to the recipient thereof.

The term “pharmaceutical composition” means a composition comprising at least one active ingredient, such as Compound A, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) inhibiting the disease; for example, inhibiting a disease, condition or disorder in an individual that is experiencing or displaying the pathology or symptomatology of the disease, condition or disorder (i.e., arresting further development of the pathology and/or symptomatology), and (2) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

### Compound 1

Compound 1 is ((2-ethylbutanoyl)oxy)methyl (R)-2-hydroxy-3-propionamido-3,4-dihydro-2H-benzo[e][1,2]oxaborinine-8-carboxylate:

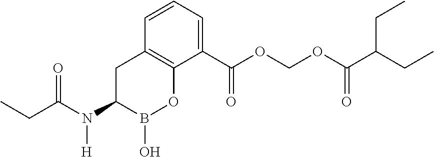 [SMILES: CCC(=O)N[C@H]1Cc2cccc(C(=O)OCOC(=O)C(CC)CC)c2OB1O]

but also exists in equilibrium with its open form (R)-(2-(3-((((2-ethylbutanoyl)oxy)methoxy)carbonyl)-2-hydroxyphenyl)-1-propionamidoethyl)boronic acid:

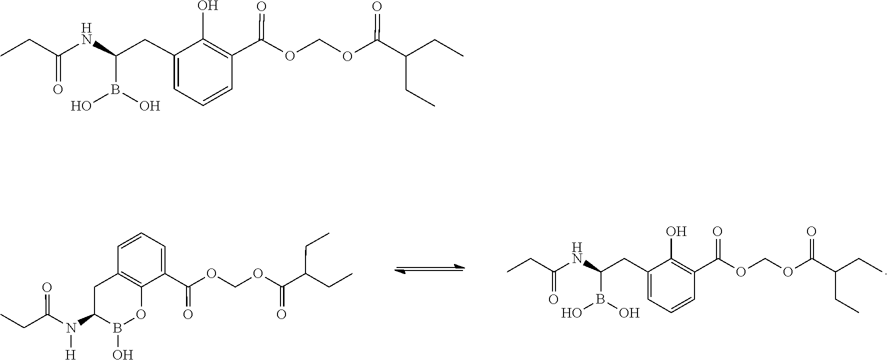 [SMILES: CCC(=O)N[C@@H](Cc1cccc(C(=O)OCOC(=O)C(CC)CC)c1O)B(O)O ; CCC(=O)N[C@H]1Cc2cccc(C(=O)OCOC(=O)C(CC)CC)c2OB1O ; CCC(=O)N[C@@H](Cc1cccc(C(=O)OCOC(=O)C(CC)CC)c1O)B(O)O]

Compound 1 is also known as ledaborbactam etzadroxil or LED-E. The synthesis of Compound 1 is disclosed in J. Med. Chem. 2021, 64, 14, 10155-10166 (Discovery of VNRX-7145 (VNRX-5236 Etzadroxil): An Orally Bioavailable β-Lactamase Inhibitor for Enterobacterales Expressing Ambler Class A, C, and D Enzymes).

### Compound 2

Compound 2 is (R)-2-hydroxy-3-propionamido-3,4-dihydro-2H-benzo[e][1,2]oxaborinine-8-carboxylic acid:

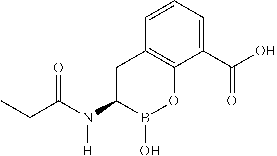 [SMILES: CCC(=O)N[C@H]1Cc2cccc(C(=O)O)c2OB1O]

and is also known as ledaborbactam or LED.

### Compound A

In some embodiments, Compound 1 exists in solid form as a covalently bound citrate coordination complex=Compound A.

In some embodiments, Compound A is (R)-2,2′-(2-(2-(3-((((2-ethylbutanoyl)oxy)methoxy)carbonyl)-2-hydroxyphenyl)-1-propionamidoethyl)-5-oxo-1,3,2-dioxaborolane-4,4-diyl)diacetic acid:

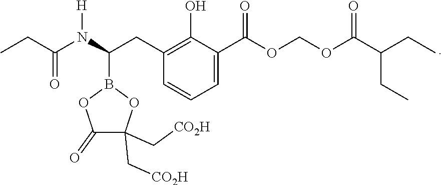 [SMILES: CCC(=O)N[C@@H](Cc1cccc(C(=O)OCOC(=O)C(CC)CC)c1O)B1OC(=O)C(CC(=O)O)(CC(=O)O)O1]

In some embodiments, the Compound A converts to Compound 1 closed form when in contact with water:

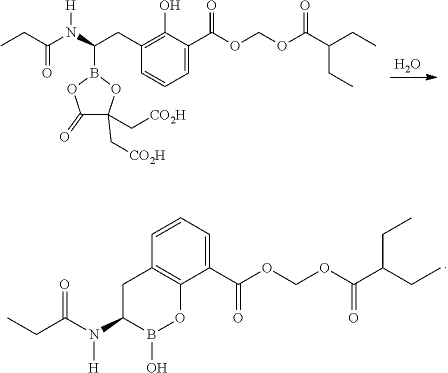

### Compound B

In some embodiments, Compound 1 exists in solid form as a covalently bound ethanolate=Compound B. In some embodiments, Compound B is ((2-ethylbutanoyl)oxy)methyl (R)-2-ethoxy-3-propionamido-3,4-dihydro-2H-benzo[e][1,2]oxaborinine-8-carboxylate:

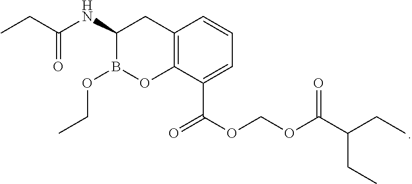 [SMILES: CCOB1Oc2c(cccc2C(=O)OCOC(=O)C(CC)CC)C[C@@H]1NC(=O)CC]

In some embodiments, the Compound B converts to Compound 1 when in contact with water:

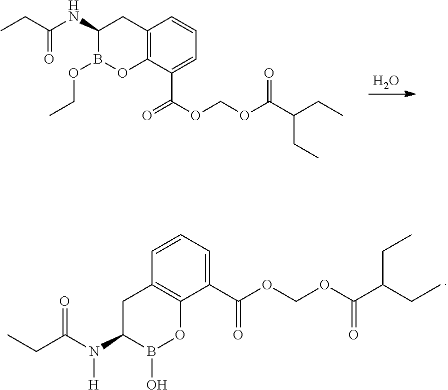

### Further Forms of Complex Disclosed Herein

In some embodiments, the amide oxygen atom of compound A may be coordinated to the boron atom in solution or in solid form and consequently the compounds described herein may be drawn (or depicted) as either

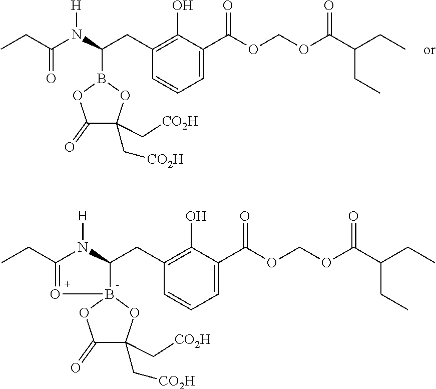

but both of them are just depictions of compound A.

Similarly, compound A may be drawn (or depicted) as either

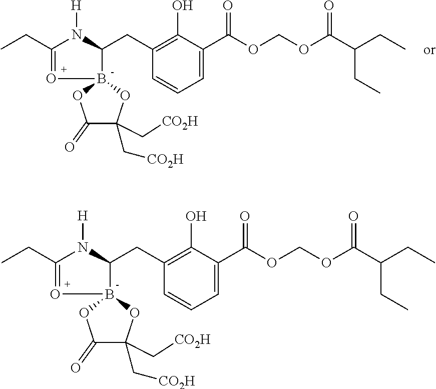

but both of them are just depictions of compound A.

Similarly, compound B may be drawn (or depicted) as either

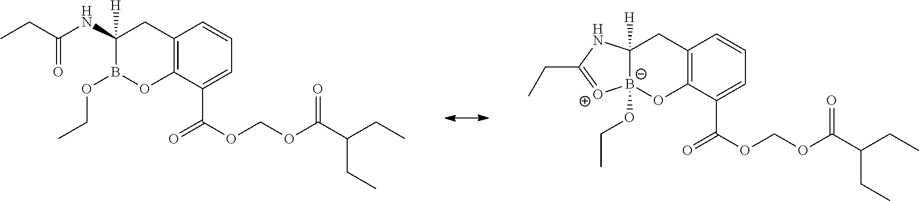 [SMILES: CCOB1Oc2c(cccc2C(=O)OCOC(=O)C(CC)CC)C[C@@H]1NC(=O)CC ; CCO[B@@-]12Oc3c(cccc3C(=O)OCOC(=O)C(CC)CC)C[C@@H]1NC(CC)=[O+]2]

but both of them are just depictions of compound B.

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center independently exists in the R configuration or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, boron, carbon, nitrogen, oxygen, phosphorous, sulfur, fluorine, and chlorine, such as ²H, ³H, ¹⁰B, ¹³C, ¹⁴C, ¹⁵N, ¹⁸O, ¹⁷O ³¹P, ³²P, ³⁵s ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, one or more hydrogen in a compound disclosed herein has been replaced by a deuterium atom. In some embodiments, one or more alkyl substituents in a compound disclosed herein has been replaced by a deuteroalkyl substituents.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, but not limited to, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, gluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁-C₄ alkyl)₄ hydroxide, and the like. In some embodiments, the compounds described herein are sodium salts. In some embodiments, the compounds described herein are disodium salts.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Methods of Treatment

Disclosed herein are methods for inhibiting bacterial growth, by, e.g., reducing bacterial resistance to a β-lactam antibiotic, such methods comprising contacting a bacterial cell culture, or a bacterially infected cell culture, tissue, or organism, with a pharmaceutical composition comprising Compound A, or a pharmaceutically acceptable salt or solvate thereof, and ceftibuten dihydrate.

Disclosed herein is a method of treating a bacterial infection in a subject in need thereof, the method comprising co-administering:
(i) a compound that is

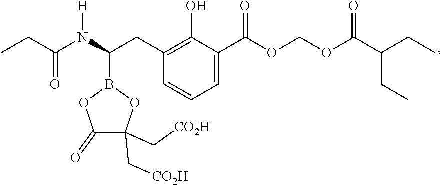 [SMILES: CCC(=O)N[C@@H](Cc1cccc(C(=O)OCOC(=O)C(CC)CC)c1O)B1OC(=O)C(CC(=O)O)(CC(=O)O)O1]

or a pharmaceutically acceptable salt or a solvate thereof, and
(ii) ceftibuten in a daily amount greater than 400 mg.

In some embodiments of a method of treating a bacterial infection, the compound is not a pharmaceutically acceptable salt or a solvate thereof.

In some embodiments of a method of treating a bacterial infection, ceftibuten is in the form of ceftibuten dihydrate.

In some embodiments of a method of treating a bacterial infection, the daily amount of ceftibuten is 400 mg to 2000 mg. In some embodiments of a method of treating a bacterial infection, the daily amount of ceftibuten is 400 mg to 1600 mg. In some embodiments of a method of treating a bacterial infection, the daily amount of ceftibuten is 400 mg to 1200 mg. In some embodiments of a method of treating a bacterial infection, the daily amount of ceftibuten is 800 mg to 2000 mg. In some embodiments of a method of treating a bacterial infection, the daily amount of ceftibuten is 1200 mg to 2000 mg. In some embodiments of a method of treating a bacterial infection, the daily amount of ceftibuten is 1600 mg to 2000 mg.

In some embodiments of a method of treating a bacterial infection, the daily amount of ceftibuten is 600 mg. In some embodiments of a method of treating a bacterial infection, ceftibuten is in the form of ceftibuten dihydrate. In some embodiments of a method of treating a bacterial infection, the daily amount of ceftibuten dihydrate administered is equivalent to 600 mg of ceftibuten. In some embodiments of a method of treating a bacterial infection, ceftibuten is administered every 12 hours (q12h) for a daily amount of 600 mg. In some embodiments of a method of treating a bacterial infection, ceftibuten is administered twice a day for a daily amount of 600 mg. In some embodiments of a method of treating a bacterial infection, ceftibuten is administered every 8 hours (q8h) for a daily amount of 600 mg. In some embodiments of a method of treating a bacterial infection, ceftibuten is administered three times a day for a daily amount of 600 mg.

In some embodiments of a method of treating a bacterial infection, the daily amount of ceftibuten is 800 mg. In some embodiments of a method of treating a bacterial infection, ceftibuten is in the form of ceftibuten dihydrate. In some embodiments of a method of treating a bacterial infection, the daily amount of ceftibuten dihydrate administered is equivalent to 800 mg of ceftibuten. In some embodiments of a method of treating a bacterial infection, ceftibuten is administered every 12 hours (q12h) for a daily amount of 800 mg. In some embodiments of a method of treating a bacterial infection, ceftibuten is administered twice a day for a daily amount of 800 mg. In some embodiments of a method of treating a bacterial infection, ceftibuten is administered every 8 hours (q8h) for a daily amount of 800 mg. In some embodiments of a method of treating a bacterial infection, ceftibuten is administered three times a day for a daily amount of 800 mg.

In some embodiments of a method of treating a bacterial infection, the daily amount of ceftibuten is 1000 mg. In some embodiments of a method of treating a bacterial infection, ceftibuten is in the form of ceftibuten dihydrate. In some embodiments of a method of treating a bacterial infection, the daily amount of ceftibuten dihydrate administered is equivalent to 1000 mg of ceftibuten. In some embodiments of a method of treating a bacterial infection, ceftibuten is administered every 12 hours (q12h) for a daily amount of 1000 mg. In some embodiments of a method of treating a bacterial infection, ceftibuten is administered twice a day for a daily amount of 1000 mg. In some embodiments of a method of treating a bacterial infection, ceftibuten is administered every 8 hours (q8h) for a daily amount of 1000 mg. In some embodiments of a method of treating a bacterial infection, ceftibuten is administered three times a day for a daily amount of 1000 mg.

In some embodiments of a method of treating a bacterial infection, the daily amount of ceftibuten is 1200 mg. In some embodiments of a method of treating a bacterial infection, ceftibuten is in the form of ceftibuten dihydrate. In some embodiments of a method of treating a bacterial infection, the daily amount of ceftibuten dihydrate administered is equivalent to 1200 mg of ceftibuten. In some embodiments of a method of treating a bacterial infection, ceftibuten is administered every 12 hours (q12h) for a daily amount of 1200 mg. In some embodiments of a method of treating a bacterial infection, ceftibuten is administered twice a day for a daily amount of 1200 mg. In some embodiments of a method of treating a bacterial infection, ceftibuten is administered every 8 hours (q8h) for a daily amount of 1200 mg. In some embodiments of a method of treating a bacterial infection, ceftibuten is administered three times a day for a daily amount of 1200 mg.

In some embodiments of a method of treating a bacterial infection, Compound A is administered in a daily amount that is equivalent to 600 mg of

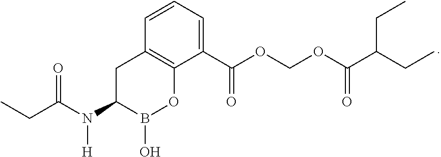 [SMILES: CCC(=O)N[C@H]1Cc2cccc(C(=O)OCOC(=O)C(CC)CC)c2OB1O]

In some embodiments of a method of treating a bacterial infection, Compound A is administered in a daily amount that is equivalent to 800 mg of

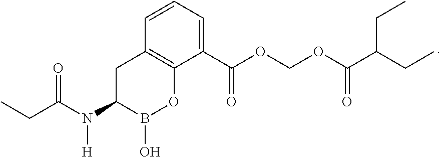 [SMILES: CCC(=O)N[C@H]1Cc2cccc(C(=O)OCOC(=O)C(CC)CC)c2OB1O]

In some embodiments of a method of treating a bacterial infection, Compound A is administered in a daily amount that is equivalent to 1000 mg of

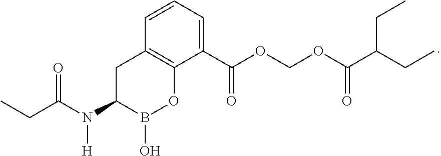 [SMILES: CCC(=O)N[C@H]1Cc2cccc(C(=O)OCOC(=O)C(CC)CC)c2OB1O]

In some embodiments of a method of treating a bacterial infection, Compound A is administered in a daily amount that is equivalent to 1200 mg of

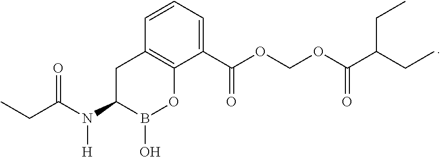 [SMILES: CCC(=O)N[C@H]1Cc2cccc(C(=O)OCOC(=O)C(CC)CC)c2OB1O]

In some embodiments of a method of treating a bacterial infection, Compound A is administered every 12 hours (q12h). In some embodiments of a method of treating a bacterial infection, Compound A is administered twice a day. In some embodiments of a method of treating a bacterial infection, Compound A is administered every 8 hours (q8h). In some embodiments of a method of treating a bacterial infection, Compound A is administered three times a day.

In some embodiments of a method of treating a bacterial infection, Compound A and ceftibuten dihydrate are administered in a ratio adjusted to correspond to between about 1:1 and about 1:4 of

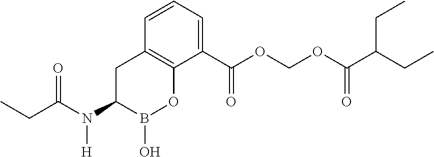 [SMILES: CCC(=O)N[C@H]1Cc2cccc(C(=O)OCOC(=O)C(CC)CC)c2OB1O]

and ceftibuten.

In some embodiments of a method of treating a bacterial infection, Compound A and ceftibuten dihydrate are administered in a ratio adjusted to correspond to between about 1:1 and about 1:3 of

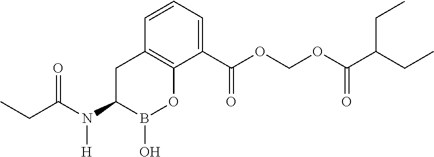 [SMILES: CCC(=O)N[C@H]1Cc2cccc(C(=O)OCOC(=O)C(CC)CC)c2OB1O]

and ceftibuten.

In some embodiments of a method of treating a bacterial infection, Compound A and ceftibuten dihydrate are administered in a ratio adjusted to correspond to between about 1:1 and about 1:2 of

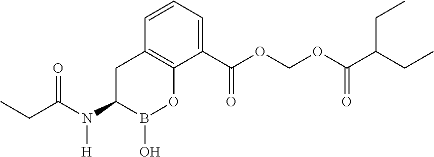 [SMILES: CCC(=O)N[C@H]1Cc2cccc(C(=O)OCOC(=O)C(CC)CC)c2OB1O]

and ceftibuten.

In some embodiments of a method of treating a bacterial infection, Compound A and ceftibuten dihydrate are administered in a ratio adjusted to correspond to between about 1:1 and about 1:1.5 of

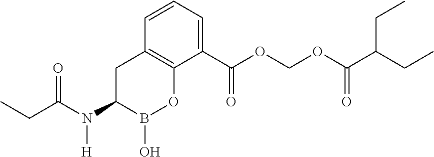 [SMILES: CCC(=O)N[C@H]1Cc2cccc(C(=O)OCOC(=O)C(CC)CC)c2OB1O]

and ceftibuten.

In some embodiments of a method of treating a bacterial infection, Compound A and ceftibuten dihydrate are administered in a ratio adjusted to correspond to about 1:4 of

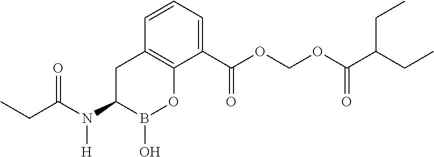 [SMILES: CCC(=O)N[C@H]1Cc2cccc(C(=O)OCOC(=O)C(CC)CC)c2OB1O]

ceftibuten.

In some embodiments of a method of treating a bacterial infection, Compound A and ceftibuten dihydrate are administered in a ratio adjusted to correspond to 1:3.5 of

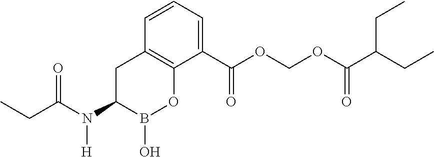 [SMILES: CCC(=O)N[C@H]1Cc2cccc(C(=O)OCOC(=O)C(CC)CC)c2OB1O]

ceftibuten.

In some embodiments of a method of treating a bacterial infection, Compound A and ceftibuten dihydrate are administered in a ratio adjusted to correspond to 1:3 of

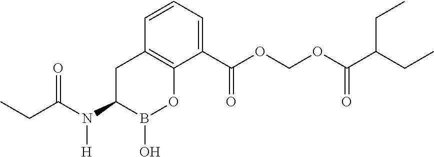 [SMILES: CCC(=O)N[C@H]1Cc2cccc(C(=O)OCOC(=O)C(CC)CC)c2OB1O]

ceftibuten.

In some embodiments of a method of treating a bacterial infection, Compound A and ceftibuten dihydrate are administered in a ratio adjusted to correspond to 1:2.5 of

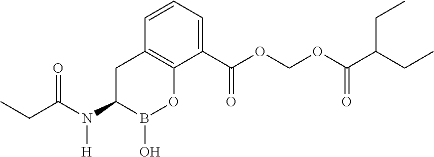 [SMILES: CCC(=O)N[C@H]1Cc2cccc(C(=O)OCOC(=O)C(CC)CC)c2OB1O]

and ceftibuten.

In some embodiments of a method of treating a bacterial infection, Compound A and ceftibuten dihydrate are administered in a ratio adjusted to correspond to about 1:2 of

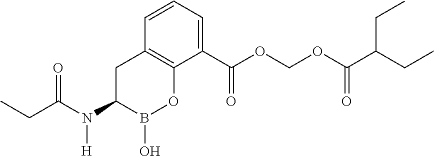 [SMILES: CCC(=O)N[C@H]1Cc2cccc(C(=O)OCOC(=O)C(CC)CC)c2OB1O]

and ceftibuten.

In some embodiments of a method of treating a bacterial infection, Compound A and ceftibuten dihydrate are administered in a ratio adjusted to correspond to about 1:1.5 of

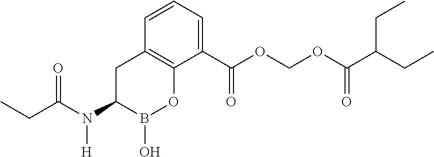 [SMILES: CCC(=O)N[C@H]1Cc2cccc(C(=O)OCOC(=O)C(CC)CC)c2OB1O]

and ceftibuten.

In some embodiments of a method of treating a bacterial infection, Compound A and ceftibuten dihydrate are administered in a ratio adjusted to correspond to about 1:1 of

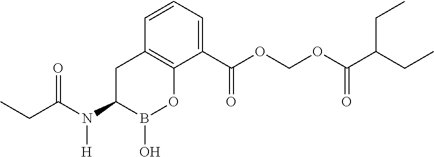 [SMILES: CCC(=O)N[C@H]1Cc2cccc(C(=O)OCOC(=O)C(CC)CC)c2OB1O]

and ceftibuten.

In some embodiments, the bacteria to be inhibited by administration of a compound described herein or a pharmaceutical composition described herein, are bacteria that are resistant to beta-lactam antibiotics. The term “resistant” is well-understood by those of ordinary skill in the art (see, e g Payne et al., Antimicrobial Agents and Chemotherapy 38 767-772 (1994), Hanaki et al., Antimicrobial Agents and Chemotherapy 30 1120-1126 (1995)).

These methods are useful for inhibiting bacterial growth in a variety of contexts. In certain embodiments, the compound described herein or the pharmaceutical composition described herein is administered to an experimental cell culture in vitro to prevent the growth of beta-lactam resistant bacteria. In certain other embodiments, the compound described herein or the pharmaceutical composition described herein is administered to a mammal, including a human, to prevent the growth of beta-lactam resistant bacteria in vivo.

Disclosed herein are methods of treating a bacterial infection, the method comprising administering to a subject a compound described herein or a pharmaceutical composition described herein.

In some embodiments, the bacterial infection is caused by carbapenem-resistant Enterobacteriaceae (CRE) or extended-spectrum beta-lactamase (ESBL) producing gram-negative bacteria.

In some embodiments, the bacterial infection is bronchitis, enteritis, gastroenteritis, gonorrhea, an intra-abdominal infection, Lyme disease, otitis media, pharyngitis, pneumonia, a respiratory tract infection, a skin infection, strep throat, tonsillitis, or a urinary tract infection.

In some embodiments, the bacterial infection is bronchitis. In some embodiments, the bronchitis is acute bacterial exacerbations of chronic bronchitis (ABECB).

In some embodiments, the bacterial infection is otitis media. In some embodiments, the otitis media is acute bacterial otitis media (AOM).

In some embodiments, the bacterial infection is a respiratory tract infection. In some embodiments, the respiratory tract infection is an upper respiratory tract infection. In some embodiments, the respiratory tract infection is a lower respiratory tract infection.

In some embodiments, the bacterial infection is a urinary tract infection. In some embodiments, the urinary tract infection is complicated urinary tract infection.

In some embodiments, the infection that is treated comprises a bacteria that includes Elizabethkingia meningoseptica, Pseudomonas aeruginosa, Pseudomonas luorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella lexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

In some embodiments, the infection that is treated comprises a bacteria that includes Escherichia coli, Citrobacter freundii, Salmonella typhimurium, Salmonella typhi, Salmonella paratyphi, Salmonella enteritidis, Shigella dysenteriae, Shigella lexneri, Shigella sonnei, Enterobacter cloacae, Enterobacter aerogenes, Klebsiella pneumoniae, Klebsiella oxytoca, Serratia marcescens, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Haemophilus infuenzae, Haemophilus parainfuenzae, Haemophilus haemolyticus, Haemophilus parahaemolyticus, Haemophilus ducreyi, Pasteurella multocida, Pasteurella haemolytica, Branhamella catarrhalis, Borrelia burgdorferi, Vibrio cholerae, Vibrio parahaemolyticus, Legionella pneumophila, Neisseria gonorrhoeae, Neisseria meningitidis, Moraxella, Streptococcus pneumoniae, Streptococcus agalactiae, and Streptococcus pyogenes.

In some embodiments, the infection that is treated comprises a bacteria that includes Elizabethkingia meningoseptica, Pseudomonas aeruginosa, Pseudomonas fuorescens, Stenotrophomonas maltophilia, Escherichia coli, Citrobacter freundii, Salmonella typhimurium, Salmonella typhi, Salmonella paratyphi, Salmonella enteritidis, Shigella dysenteriae, Shigella fexneri, Shigella sonnei, Enterobacter cloacae, Enterobacter aerogenes, Klebsiella pneumoniae, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus infuenzae, Haemophilus parainfuenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella, Bacteroides fragilis, Bacteroides vulgatus, Bacteroides ovalus, Bacteroides thetaiotaomicron, Bacteroides uniformis, Bacteroides eggerthii, or Bacteroides splanchnicus.

In some embodiments, the infection that is treated comprises a bacteria that includes Escherichia coli, Citrobacter freundii, Salmonella typhimurium, Salmonella typhi, Salmonella paratyphi, Salmonella enteritidis, Shigella dysenteriae, Shigella fexneri, Shigella sonnei, Enterobacter cloacae, Enterobacter aerogenes, Klebsiella pneumoniae, Klebsiella oxytoca, Serratia marcescens, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Vibrio cholerae, Vibrio parahaemolyticus, Legionella pneumophila, Listeria monocytogenes, Neisseria gonorrhoeae, and Neisseria meningitidis, Moraxella.

### EXAMPLES

### Example 1: Preparation of ((2-ethylbutanoyl)oxy)methyl (R)-2-ethoxy-3-propionamido-3,4-dihydro-2H-benzo[e][1,2]oxaborinine-8-carboxylate (Compound B) from Compound 1

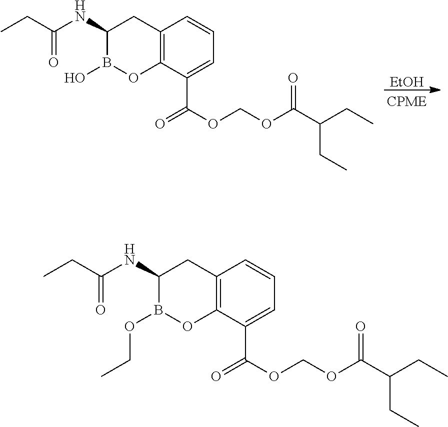

A solution of ((2-ethylbutanoyl)oxy)methyl (R)-2-hydroxy-3-propionamido-3,4-dihydro-2H-benzo[e][1,2]oxaborinine-8-carboxylate (Compound 1—J. Med. Chem. 2021, 64, 14, 10155-10166) in 18 volumes of cyclopentyl methyl ether (CPME) was distilled under vacuum to until the volume was reduced to 7.5 volumes while maintaining a temperature below ≤65° C. A Karl Fischer (KF) titration was performed to ensure the level of residual water was reduced to a value below 5000 ppm. The solution was diluted to 17 volumes with additional CPME followed by 6 volumes of ethanol. The solution was distilled under vacuum until a volume of ~7.5 volumes was achieved. The composition of the solution was examined to ensure that the level of residual water was below 1000 ppm by KF and the ethanol content was between 6 and 11 volume % determined using ¹H NMR spectroscopy. The temperature was raised to 55±5° C. over 43 min. and methylcyclohexane (22.5 volumes) added over 50 min while maintaining the temperature at 55±5° C. The temperature was adjusted to 45±5° C. and the mixture seeded with 0.15% by weight ((2-ethylbutanoyl)oxy)methyl (R)-2-ethoxy-3-propionamido-3,4-dihydro-2H-benzo[e][1,2]oxaborinine-8-carboxylate seeds Form A. After addition of the seeds solid formation was observed. The slurry was stirred at 45±5° C. for approximately 4 h, then cooled to 20±5° C. over ~2.5 h and stirred for an additional 11 h at 20±5° C. The product was isolated by filtration under a nitrogen. The solid was washed with methylcyclohexane (7 volumes) over approximately a 7 h period. The resulting wet cake was dried at 30° C. under vacuum to constant weight over about 68 h. The above process provided ((2-ethylbutanoyl)oxy)methyl (R)-2-ethoxy-3-propionamido-3,4-dihydro-2H-benzo[e][1,2]oxaborinine-8-carboxylate (Compound B), Form A, in 79% with a purity of 99.7 AUC % by Ultra-High Performance Liquid Chromatography (UHPLC).

### Example 2: Preparation of Compound A, Form A, from Compound B

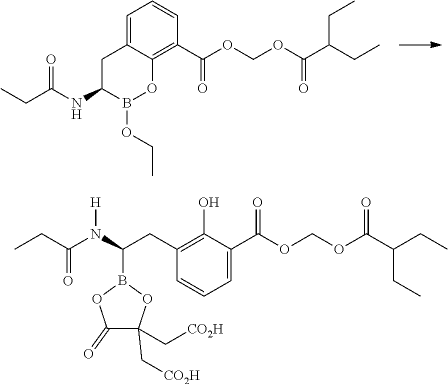

2.105 g of Compound B and 0.968 g of citric acid (1:1 molar ratio) were added into a 100 mL round-bottom flask with 15 mL of anhydrous ethyl acetate. The resulting solution was heated and maintained at 80° C. for 3.5 hours. Inert atmosphere (N₂ atmosphere) was maintained throughout the reaction. The resultant solution from step-1 was evaporated. The obtained glue like material was further dried by purging with N₂ for ~15 hours. The resultant solid from Step-2 was treated with 20 mL of diethyl ether and stirred for 1 hour. 20 mL of n-heptane was added to that suspension and stirred for another 24 hours. The solids obtained were filtered and dried at room temperature for ~2 hours.

### Example 3: Preparation of Compound A, Form A, from Compound 1

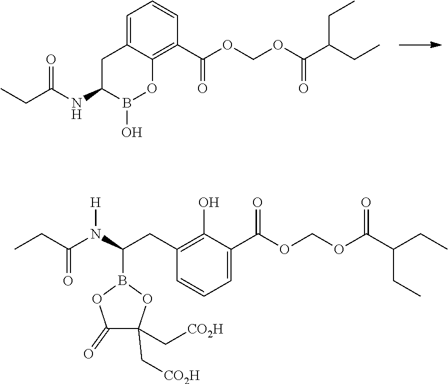

((2-Ethylbutanoyl)oxy)methyl (R)-2-hydroxy-3-propionamido-3,4-dihydro-2H-benzo[e][1,2]oxaborinine-8-carboxylate (Compound 1—J. Med. Chem. 2021, 64, 14, 10155-10166, 125 grams, 1 eq, 95.0 w/w %) was dissolved in acetonitrile (1250 ml, 10 volumes) followed by addition of citric acid (anhydrous, 54.4 g, 1 eq) to generate a heterogenous solution. The mixture was agitated at ambient temperature for two hours or until a homogenous solution was formed.

Acetonitrile was distilled under vacuum to 3-4 volumes followed by addition of 10 volumes of isopropyl acetate. The batch was distilled, under vacuum, to 3-4 volumes followed by addition of 10 volumes of isopropyl acetate two more times or until the water content was below 1000 ppm. The volume of the batch was adjusted to 9-10 volumes of isopropyl acetate and then heated to 65° C. The batch was cooled to 45-50° C. and seeded with 0.1 w/w % Compound A, Form A, (from example 2) and held for 30 min for crystals to form. Once a seed bed formed, the batch was cooled to 20-25° C. and held for 16 h. The batch was cooled to 5° C., held for 2 h, and then filtered, and washed with cold (5° C.) isopropyl acetate/heptane (3×3v, 3/1 v/v). The solid was dried on the filter and then under vacuum to constant weight to yield 144 grams of a white solid. The process above provided Compound A, Form A, in 90% yield and UHPLC purity of 99.9% and a qNMR assay purity of 99.3 w/w % (ethylene carbonate as internal standard). The mass spectrum included peaks with m z 564 for the [M-H]⁻, and m z 588 for the [M+Na]⁺ peak, in agreement with the monoisotopic mass of the proposed molecular formula of C₂₅H₃₂BNO₁₃. The molecular mass of Compound A, Form A is 565.34 Da and the exact mass is 565.20 Da.

The 1D proton spectrum showed the expected chemical shifts, multiplicities and integrations that are consistent with the structure of Compound A. Duplicate ¹H and ¹³C resonances were observed (due to the presence conformational isomers of the amide functionality). The data are summarized in the table below.

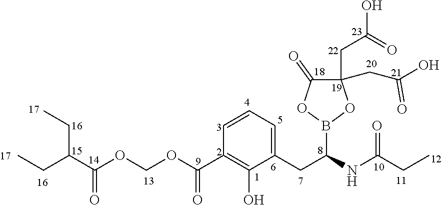 [SMILES: CCC(=O)N[C@@H](Cc1cccc(C(=O)OCOC(=O)C(CC)CC)c1O)B1OC(=O)C(CC(=O)O)(CC(=O)O)O1]

| Position | Proton Shift | | Proton Multiplicity | Carbon Shift ^{§} |
|---|---|---|---|---|
| No. | (ppm) | Integration | (J in Hz) | (ppm) |
| 1 | N/A | N/A | N/A | 161.3 |
| 2 | N/A | N/A | N/A | 112.02, 111.99^{§} |
| 3 | 7.71 | 1 | d (J = 8.0) | 128.9 |
| 4 | 6.90 | 1 | t (J = 7.7) | 120.2 |
| 5 | 7.52 | 1 | d (J = 7.4) | 138.6, 138.4^{§} |
| 6 | N/A | N/A | N/A | 130.7, 130.6^{§} |
| 7 | 3.05, 2.78, | 2 | m | 31.2, 30.9^{§} |
| | 2.96, 2.71 | | | |
| 8 | 3.10, 3.04 | 1 | m | 48.4 (br) |
| 9 | N/A | N/A | N/A | 170.1 |
| 10 | N/A | N/A | N/A | 182.2, 182.0^{§} |
| 11 | 2.56 | 2 | m | 24.38, 24.36^{§} |
| 12 | 1.20, 1.18 | 3 | t (J = 7.5), t (J = 7.4) | 9.4 |
| 13 | 6.10, 6.093, | 2 | | 80.68, 80.66^{§} |
| | 6.091, 6.08 | | | |
| 14 | N/A | N/A | N/A | 174.9 |
| 15 | 2.32 | 1 | m | 49.2 |
| 16 | 1.60 | 4 | m | 25.47, 25.46^{§} |
| 17 | 0.89 | 6 | t (J = 7.5) | 11.9 |
| 18 | N/A | N/A | N/A | 178.4, 178.3^{§} |
| 19 | N/A | N/A | N/A | 77.7, 77.5^{§} |
| 20 | 2.74, 2.72 | 2 | m | 43.7, 43.8^{§} |
| 21 | N/A | N/A | N/A | 170.7, 170.6^{§} |
| 22 | 3.09, 2.99, | 2 | m | 41.8, 41.7^{§} |
| | 2.92, 2.80 | | | |
| 23 | N/A | N/A | N/A | 171.4, 171.2^{§} |
| 1-OH | 10.74, 10.72 | 2.76^{¥} | s | N/A |
| 8-NH-10 | 9.43 | 1.0 | d (J = 9.2) | N/A |
| 21, 23-OH | 10.65 | 2.76^{¥} | br s | N/A |

s = singlet, t = triplet, q = quartet, dt = doublet of triplets, m = multiplet, br = broad, ppm = parts per million.
NA = Not Applicable.
^{¥}Integration for 1-OH and 21, 23-OH.
^{§}The splitting of ¹³C resonances of C2, C5, C6, C7, C10, C11, C13, C16, C18, C19, C20, C21, C22 and C23 is due to the presence of conformational isomers.
^{#}The two CH₂CO₂H of the boron-citric acid coordination complex are in non-equivalent environments.

### Example 4: Safety and Pharmacokinetics Study of Ceftibuten (CTB) in Healthy Adults at Higher does

Ledaborbactam etzadroxil, the prodrug of the active β-lactamase inhibitor ledaborbactam, is being developed in combination with ceftibuten (CTB), to treat serious infections caused by drug-resistant Enterobacterales. This study evaluated the safety and pharmacokinetics of ceftibuten in healthy adults at anticipated higher doses required, in combination with ledaborbactam etzadroxil, to treat Enterobacterales infections. Thirty-six participants, (n=12 per cohort [ceftibuten n=9, placebo n=3]) received a single oral dose of ceftibuten (400, 800, or 1200 mg) or matched placebo on Day 1. Following a one-day washout, the same participants received repeat oral doses of ceftibuten (400 mg once daily, 400 mg every 12 hours [q12 h], or 400 mg q8h) for 10 days. Of the 36 participants, 25 (ceftibuten 67%, placebo 78%) reported 65 treatment-emergent adverse events (TEAEs). Nausea (22%), headache (15%), and fatigue (15%) were the most reported TEAEs among ceftibuten-treated participants. No participant experienced serious adverse events or discontinuations due to TEAEs. In both single- and multiple-dose cohorts, cis-ceftibuten isomer exposure was dose-proportional for areas under the curve (AUCs) but less than dose proportional for maximum concentrations (Cₘₐₓ). Low levels of cis-ceftibuten accumulation were observed at steady state, with the highest accumulation ratio of 1.24 in the 400 mg q8 h cohort. Cis-ceftibuten and trans-ceftibuten recovery in urine was 47% and 6% following a 1200 mg single dose, respectively.

### Study Design

This phase 1, two-period, randomized, double-blind, placebo-controlled, sequential-group, ascending dose study of ceftibuten administered orally for 10 days in healthy adults was conducted at a single site. The ceftibuten used was commercially available ISOCEF™ 400 mg capsules (marketed in Italy by Recordati). In addition to ceftibuten the capsules contain microcrystalline cellulose, sodium starch glycolate, magnesium stearate. The capsules are hard gelatin capsules size 00.

Eligible participants were randomized at a 3:1 ratio to receive ceftibuten or placebo. Participants were enrolled into 1 of 3 cohorts; each cohort comprised 12 participants, 9 randomized to ceftibuten and 3 randomized to placebo. Within each cohort, a single oral dose was administered on Day 1 (single dose period), followed by a 1-day washout period, and 10 days of repeated oral doses (Days 3-12; multiple dose period). Participants in the 400 mg cohort received a single dose of ceftibuten 400 mg or placebo during the single dose period and ceftibuten 400 mg or placebo once daily during the multiple dose period. Participants in the 800 mg cohort received a single dose of ceftibuten 800 mg or placebo during the single dose period and ceftibuten 400 mg every 12 hours (q12h; total daily dose 800 mg) or placebo during the multiple dose period. Participants in the 1200 mg cohort received ceftibuten 1200 mg or placebo during the single dose period and ceftibuten 400 mg q8h (total daily dose 1200 mg) or placebo during the multiple dose period. On Day 12, participants in the 400 mg q12 h and 400 mg q8 h cohorts received only a single morning dose.

After completion of the 400 mg and 800 mg cohorts, a safety monitoring committee reviewed all available blinded safety data before the decision was made to begin dosing in the 1200 mg cohort.

The safety findings of this study indicated that single doses of ceftibuten up to 1200 mg and multiple doses up to 400 mg q8 h for 10 days were safe and well tolerated, with a consistent safety profile at doses exceeding the approved 400 mg daily dose.

The vast majority of ceftibuten that is dosed is cis-ceftibuten and following dosing, ceftibuten undergoes isomerization to trans-ceftibuten; approximately 10% of ceftibuten is converted to the trans-ceftibuten isomer, which has about one-eighth the antimicrobial potency of the cis-ceftibuten isomer. Consistent with the known pharmacokinetic characteristics of ceftibuten, in our study cis-ceftibuten was the predominant isomer in plasma; exposures of the trans-ceftibuten isomer represented <10% of the total exposure. The current findings generally align with the prior studies of single and multiple doses of ceftibuten 400 mg, in which mean cis-ceftibuten AUC has been reported as 73.7-95.3 mg·h/mL, Cₘₐₓ as 15.0-17.6 mg/mL, Tₘₐₓ as 2.0-2.6 h, and t_{1/2} as 2.3-2.5 h (Lin et al. Antimicrobial Agents and Chemotherapy 39:356-358 (1995), Hernández-Mitre et al., Antimicrobial Agents and Chemotherapy 68:e0109923 (2024), Bressolle et al., Journal of Pharmaceutical Sciences 83(9):1236-40 (1994), Lin et al., Antimicrobial Agents and Chemotherapy 39:359-361 (1995), Barr et al. Diagnostic Microbiology and Infectious Disease 14:93-100 (1991)). Plasma protein binding was also similar to previously reported values. Dose proportionality was observed for AUC but was less than dose proportional for Cₘₐₓ following single doses of 400-1200 mg.

Prior studies in healthy adults have evaluated pharmacokinetics and safety in single doses up to 800 mg and multiple doses up to 800 mg twice daily. Similar to the current findings, the earlier studies demonstrated acceptable oral bioavailability, with Cₘₐₓ reached within 2-3 hours after single doses and a t_{1/2} of 1-3 hours following single and multiple doses.

About 60%-90% of the drug is recovered as cis-ceftibuten in urine after a single dose. Total urinary recovery within 24 hours of the administered ceftibuten dose was approximately 53% in the 1200 mg cohort. In one recent study, urinary recovery, as cis-ceftibuten, as high as 83% and 87% was observed following single doses of ceftibuten 400 mg and 600 mg, respectively, but was 64% after a single dose of ceftibuten 800 mg. The lower urinary recovery, as cis-ceftibuten, of 47% following a single dose of ceftibuten 1200 mg in the current study may be due in part to a shorter collection period relative to the study by Hemández-Mitre et al., Antimicrobial Agents and Chemotherapy 68(1):e e0109923 (2024) (collection over 24 hours versus 48 hours). In a study by Bressolle et al., Journal of Pharmaceutical Sciences 83(9):1236-40 (1994) in which participants received ceftibuten 400 mg qP2 h for 7 days, urinary recovery of 58% at 12 hours following a single dose ofceftibuten had increased to 634 at 48 hours following the Day 8 dose. In addition, lower recovery at a 1200 mg dose might reflect an elimination pathway that is subject to saturation at higher doses; this is supported by the findings from Hem indez-Mitre et al.

In conclusion, this study in healthy adults demonstrated that higher doses of ceftibuten were safe and well tolerated. Ceftibuten is a highly bioavailable oral cephalosporin and the pharmacokinetic profile at higher doses is favorable and supports the higher doses required to treat drug-resistant Enterobacterales infections. This study supports the continued evaluation of ceftibuten-ledaborbactam etzadroxil as an oral treatment for serious infections including complicated urinary tract infections (cUTI).

TABLE 1
Geometric mean (% geometric coefficient of variation) cis-ceftibuten
and trans-ceftibuten pharmacokinetic parameters following single-dose
(Day 1) and multiple-dose (Day 12) oral administration of ceftibuten

| | | | | | | Ceftibuten | |
|---|---|---|---|---|---|---|---|
| | Pharmacokinetic | Ceftibuten 400 | | Ceftibuten 800 | | 1200 mg | |
| Analyte | Parameter^{a} | mg Cohort | | mg Cohort | | Cohort | |
| | | 400 mg | | 800 mg | | 1200 mg | |
| Day 1^{b} | | (n = 9) | | (n = 9) | | (n = 9) | |
| Cis-ceftibuten | Cₘₐₓ (μg/mL) | 16.9 | (14.5) | 27.5 | (18.4) | 35.8 | (16.3) |
| | Tₘₐₓ^{c} | 2.5 | (1.5-4.0) | 2.1 | (1.5-4.0) | 2.5 | (1.5-4.0) |
| | t_{1/2} (h) | 2.9 | (22.0) | 2.8 | (23.5) | 2.7 | (22.7) |
| | AUC_{inf} (h · μg/mL) | 75.5 | (21.0) | 143 | (21.1) | 203 | (22.0) |
| Trans-ceftibuten | Cₘₐₓ (μg/mL) | 0.753 | (18.7) | 1.45 | (26.1) | 1.75 | (18.9) |
| | Tₘₐₓ^{c} | 4.0 | (2.0-5.0) | 4.0 | (2.5-5.0) | 4.0 | (3.0-5.0) |
| | t_{1/2} (h) | 3.52 | (21.1) | 3.46 | (15.6) | 3.30 | (11.5) |
| | AUC_{inf} (h · μg/mL) | 5.64 | (22.4) | 11.0 | (26.5) | 14.2 | (26.9) |
| | | 400 mg once | | | | | |
| | | daily | | 400 mg q12h^{e} | | 400 mg q8h^{e} | |
| Day 12^{d} | | (n = 9) | | (n = 9) | | (n = 8) | |
| Cis-ceftibuten | Cₘₐₓ (μg/mL) | 18.2 | (19.1) | 19.1 | (15.6) | 24.7 | (15.5) |
| | Tₘₐₓ^{d} | 2.0 | (1.5-4.0) | 3.0 | (2.0-5.0) | 3.0 | (2.0-4.0) |
| | t_{1/2} (h) | 2.68 | (14.2) | 2.52 | (18.0) | 2.80 | (26.7) |
| | AUCₜₐᵤ (h · μg/mL) | 82.4 | (19.3) | 85.9 | (21.0) | 105 | (16.3) |
| | R_{ac}^{f} | 1.06 | (7.0) | 1.09 | (18.8) | 1.24 | (14.4) |
| Trans-ceftibuten | Cₘₐₓ (μg/mL) | 0.756 | (21.1) | 0.970 | (20.7) | 1.28 | (27.4) |
| | Tₘₐₓ^{c} | 4.0 | (2.7-6.0) | 4.0 | (3.0-6.0) | 4.0 | (3.0-5.0) |
| | t_{1/2} (h) | 3.57 | (13.1) | 3.44 | (13.3) | 3.61 | (12.9) |
| | AUCₜₐᵤ (h · μg/mL) | 5.80 | (23.2) | 6.25 | (23.4) | 7.27 | (28.8) |
| | R_{ac}^{f} | 1.01 | (8.3) | 1.22 | (24.4) | 1.56 | (19.0) |

^{a}AUC_{inf}, area under the plasma concentration-time curve from time 0 through infinity; AUCₜₐᵤ, area under the concentration-time curve from time of administered dose through dosing interval (daily, tau = 24 hours; q12h, tau = 12 hours; q8h: tau = 8 hours); Cₘₐₓ, maximum plasma concentration; t_{1/2}, terminal half-life.
^{b}Parameters assessed following a single dose.
^{c}Tₘₐₓ values are median (range).
^{d}Parameters assessed on Day 12, after 10 consecutive days of dosing.
^{e}q12h, every 12 hours; q8h, every 8 hours.
^{f}R_{ac} calculated as AUCₜₐᵤ Day 12/Day3.

### Example 5: Phase 1 Studies with LED-E and Ceftibuten (LED-E-CTB)

Described herein are the results from two Phase 1 studies from the CTB+LED-E drug development program. The first-in-human study VNRX-7145-101 (NCT04243863) focused on the safety and pharmacokinetics following single and multiple oral doses of LED-E (administered as Compound B) in healthy participants. VNRX-7145-102 (NCT04877379) expanded on this by evaluating the potential for drug-drug interactions between CTB and LED and the safety and pharmacokinetics of multiple doses of the CTB+LED-E (administered as Compound B) combination in healthy participants.

### Safety and Tolerability

The number of participants with treatment-emergent adverse events (TEAE) was similar in the LED-E±CTB (81.7%) and placebo (77.8%) groups. Among LED-E-dosed participants, headache (20.2%) and fatigue (9.2%) were the most frequently reported TEAEs. Gastrointestinal TEAEs occurred in 27.5% of LED-E-dosed participants and 14.8% of placebo-dosed participants, were mild, and did not lead to discontinuation of study drug. One participant in the LED-E 150 mg q8 h group discontinued the study due to a mild, related TEAE of noncardiac chest pain. No serious adverse events or deaths occurred. No clinically relevant changes were noted in clinical laboratory values, ECGs, or vital signs.

### Pharmacokinetics

### Single Doses of LED-E

### Plasma Results

Pharmacokinetic parameters were assessed following a single dose of LED-E 100 to 1000 mg. LED concentrations increased rapidly after administration, reaching Cₘₐₓ at 1.25 to 2 hours post-dose, then decreased in a biphasic manner over time. LED exposure generally increased dose proportionally, with geometric mean AUC_{inf} values of 15,400 h·ng/mL (LED-E 100 mg) to 114,000 h·ng/mL (LED-E 1000 mg). LED Cₘₐₓ and AUC_{inf} exhibited low variability across the dose range (Table 2).

Following single doses of LED-E 100 to 1000 mg, the AUC_{inf} of LED-E was <2% of LED exposures, suggesting extensive presystemic conversion of the prodrug to the active drug. Exposure generally increased in a dose-proportional manner across the dose range, from a geometric mean AUC_{inf} of 177 h·ng/mL (LED-E 100 mg) to 2300 h·ng/mL (LED-E 1000 mg). Geometric mean plasma concentrations increased rapidly following single doses of LED-E with maximum plasma concentrations (Cₘₐₓ) reached 0.5 to 0.75 hours post-dose (Table 2). The geometric mean t_{1/2} was 2.8 to 4.7 hours, with no clear trend as dose increased.

Co-administration of a single dose of CTB 400 mg with LED-E 500 mg had no clinically relevant impact on LED, LED-E, cis-CTB, or trans-CTB plasma concentrations. For LED concentrations, the effect on Cₘₐₓ (Geometric Mean Ratio (GMR), 0.96 [90% CI, 0.89 to 1.03]) and AUC_{inf} (GMR, 0.99 [90% CI, 0.95 to 1.03]) indicated an absence of interaction. An interaction cannot be ruled out for LED-E Cₘₐₓ (GMR, 0.76 [90% CI, 0.66 to 0.87]; indicating 24% decrease in LED-E Cₘₐₓ with coadministration) but an absence of interaction was observed for AUC_{inf} (GMR, 0.94 [0.85 to 1.04]) (Table 4). An interaction is unlikely but cannot be ruled out for cis-CTB Cₘₐₓ (GMR, 0.88 [0.79 to 0.97]), but an absence of interaction was observed for AUC_{inf} (GMR, 0.88 [0.81 to 0.95]). For trans-CTB concentrations, an interaction is unlikely but cannot be ruled out for Cₘₐₓ (GMR, 0.85 [90% % CI, 0.77 to 0.94]) and AUC_{inf} (GMR, 0.86 [90% CI, 0.79 to 0.93]).

### Multiple Doses of LED-E

### Plasma Results

Pharmacokinetic parameters were assessed following 10 days of dosing with LED-E 75 to 500 mg q8h. On Day 10, LED concentrations increased rapidly after administration, reaching Cₘₐₓ at 0.75 to 1.50 hours postdose (Table 3). LED exposure generally increased dose proportionally, with geometric mean AUCₜₐᵤ (0 to 8 hours postdose) of 13,900 h·ng/mL (LED-E 75 mg q8h) to 69,988 h·ng/mL (LED-E 500 mg). For all dose levels assessed, AUCₜₐᵤ and Cₘₐₓ increased approximately 30% to 35% from the first to last dose. The Day 10 t_{1/2} was 9.3 to 12.5 h across dose groups. Steady state LED plasma concentrations appeared to be reached by Day 3.

Plasma concentrations of LED-E also increased rapidly following multiple doses of LED-E 75 to 500 mg q8h. Exposure increased with increasing dose, from a geometric mean AUCₜₐᵤ of 85.5 h·ng/mL (LED-E 75 mg q8h) to 1048 h·ng/mL (LED-E 500 mg q8h). Geometric mean Cₘₐₓ was reached 0.5 to 0.75 hours post-dose on Day 10, and increased from 122 ng/mL (LED-E 75 mg q8h) to 919 ng/mL (LED-E 500 mg q8h). The geometric mean t_{1/2} increased with dose, from 3.07 h (LED-E 75 mg q8h) to 9.14 h (LED-E 500 mg q8h).

On Day 10 following multiple doses of CTB+LED, the geometric mean LED AUCₜₐᵤ increased 24% from the first dose (26,968 h·ng/mL to 33,319 h·ng/mL) in the CTB 400 mg+LED-E 300 mg q8 h cohort and 26% (49,485 h·ng/mL to 62,239 h·ng/mL) in the CTB 400 mg+LED-E 500 mg q8 h cohort. In both dose groups, the LED-E geometric mean AUCₜₐᵤ was unchanged from the first to last dose. The geometric mean AUCₜₐᵤ for cis-CTB increased 44% from the first dose (64,585 h·ng/mL to 92,982 h·ng/mL) in the CTB 400 mg+LED-E 300 mg q8 h cohort and 52% (68,826 h·ng/mL to 104,817 h·ng/mL) in the CTB 400 mg+LED-E 500 mg q8 h cohort. The geometric mean AUCₜₐᵤ for trans-CTB increased 64% from the first dose (5333 h·ng/mL to 8734 h·ng/mL) in the CTB 400 mg+LED-E 300 q8 h group and 75 (5781 h·ng/mL to 10,138 h·ng/mL) in the CTB 400 mg+LED-E 500 q8 h group.

### DISCUSSION

The combination has demonstrated potent in vitro and in vivo activity against drug-resistant Enterobacterales, including strains producing ESBLs, AmpC, and serine carbapenemases such as KPC and OXA-48. LED restores CTB susceptibility, reducing MIC values by 32- to 1024-fold, with MIC90 values ranging from <0.25 to <2 μg/mL, depending on the β-lactamase type, including CTX-M-1, CTX-M-9, and SHV ESBLs. In in vitro studies, over 98% of ESBL-positive isolates were inhibited at MICs ≤1 μg/mL, and the combination showed significant efficacy against KPC (85.9%) and OXA-48 (82.9%) producers.

In vivo studies using a human-simulated regimen of CTB+LED in neutropenic murine models confirmed bacterial stasis in 20 of 21 strains, while CTB monotherapy resulted in increased bacterial burden (14). Comparisons with other β-lactam/0-lactamase inhibitor combinations, such as ceftazidime-avibactam and meropenem-vaborbactam, revealed superior or comparable activity, particularly against carbapenem-resistant isolates.

In a study of 3,889 global isolates, 89.7% of drug-resistant isolates were inhibited at <1 μg/mL, including robust activity against CTX-M, KPC, and OXA-48 producers. LED restored the efficacy of CTB against carbapenemase-producing Klebsiella pneumoniae, with 92.5% of isolates susceptible under EUCAST criteria compared to only 4.5% for CTB alone (Karlowsky et al.

### Antimicrobial Agents and Chemotherapy 62(9):66(11) (2022)).

The current findings support the continued development of this oral combination as an effective carbapenem-sparing therapeutic option for treating cUTIs and other serious infections caused by MDR pathogens, potentially reducing the need for intravenous therapies, hospitalizations, and healthcare costs. LED-E was well tolerated at all dose levels evaluated when dosed alone or in combination with CTB, with no serious or severe TEAEs following single or multiple doses. Gastrointestinal symptoms were the most common TEAE reported for the active intervention groups. The most frequent system organ class among the reported TEAEs, not including investigations, was gastrointestinal disorders. Participants receiving CTB 400 mg+LED-E (300 mg or 500 mg) q8 h for 10 days had a higher frequency of frequent bowel movements (7/20; 35%) but not diarrhea (1/20; 5%). Ascending single doses (100 to 1000 mg) and multiple doses (75 to 500 mg) of LED-E showed no dose relationship with the frequency or severity of TEAEs.

Pharmacokinetic analysis of single doses of LED-E revealed a dose-proportional increase in plasma levels of LED as the LED-E dose increased from 100 to 1000 mg. LED exposure also remained dose proportional following 10 days of q8 h dosing. AUCₜₐᵤ increased approximately 30% to 35% from the first to last doses, indicating moderate accumulation of LED in plasma after 10 days of dosing; and steady-state plasma concentrations were reached by Day 3. These findings confirmed rapid conversion to the active inhibitor and supported the feasibility of LED-E as an oral therapy. Urinary excretion, almost entirely as LED, is the primary route of elimination following oral dosing of LED-E (84% of the drug was excreted in urine during the dosing interval in the 500 mg q8 h dosing regimen), supporting its potential role in the treatment of cUTI.

Plasma pharmacokinetics results were similar between cohorts that received multiple doses of the CTB+LED-E combination and those that received LED-E alone. For example, the LED-E 500 mg q8 h cohort had a Cₘₐₓ of 15,788 ng/mL and the CTB 400 mg+LED-E 500 mg q8 h cohort had a Cₘₐₓ of 16,152 ng/mL; the respective AUCₜₐᵤ values were 69,988 and 62,239 h·ng/mL (Table 3). Assessments of the pharmacokinetic interactions between CTB and LED-E suggested borderline statistically significant interactions for cis- and trans-CTB Cₘₐₓ and AUC and statistically significant interactions for LED-E Cₘₐₓ. The effects on the prodrug Cₘₐₓ and CTB exposure parameters are not considered to be clinically relevant supporting the further development of the combination. In conclusion, the findings from these Phase 1 studies support the further development of the CTB+LED-E combination for cUTI caused by drug-resistant Enterobacterales. The overall safety profile of the CTB+LED-E combination was consistent with what has been observed with CTB (ISOCEF (ceftibuten) [summary of product characteristics]. 2007. Milan, Italy RECORDATI Industria Chimica e Farmaceutica S.p.A; CEDAX (ceftibuten) [product information]. 2010. Pernix Therapeutics, LLC, Gonzales, LA) and the pharmacokinetic profile of LED is favorable for co-administration with CTB.

### Study Design

VNRX-7145-101 was a 2-part randomized, double-blind, placebo-controlled, sequential-group study of the safety and pharmacokinetics of single (Part 1) and multiple (Part 2) ascending doses of LED-E in 48 healthy adults.

VNRX-7145-102 was a 3-part randomized study of CTB and LED-E in healthy adults. Part 1 used an open-label, cross-over design to evaluate safety and drug-drug interaction (DDI) of a single dose of LED-E 500 mg, CTB 400 mg, and CTB+LED-E combination, in 18 participants randomized in a 1:1:1:1:1:1 sequence ratio. Part 2 evaluated multiple doses of LED-E 500 mg or placebo q8h, administered for 10 days, in 12 participants randomized in a 3:1 ratio. Part 3 used a double-blind, parallel-group design to evaluate safety and pharmacokinetics of CTB 400 mg+LED-E 300 mg, CTB 400 mg+LED-E 500 mg, or placebo q8 h for 10 days, in 24 participants randomized in a 5:5:2 ratio. Part 3 was designed to enroll 24 participants, but enrollment was halted at 23 participants due to slow enrollment at the clinical site.

In both the VNRX-7145-101 and VNRX-7145-102 studies LED-E was administered as the Self-Emulsifying Drug Delivery System (SEDDS) in white opaque hydroxypropyl methylcellulose (HPMC) size 0 capsules (see Example 10 for formulation preparation). Although LED-E was administered as Compound B, all indicated mg amounts indicate the equivalent mg amounts of LED-E Compound 1. In both the VNRX-7145-101 and VNRX-7145-102 studies CTB was administered orally as the ISOCEF™ 400 mg capsules (see Example 4).

### Assessments

In both studies, safety was assessed via ongoing monitoring of adverse events, symptom-directed physical examinations, and changes in clinical laboratory measures, vital signs, and ECGs.

For determination of single-dose and steady-state plasma pharmacokinetics, serial blood samples were collected at the following timepoints in VNRX-7145-101: in the SAD part (Part 1), pre-dose and 0.25, 0.5, 0.75, 1, 1.5, 2, 2.5, 3, 4, 5, 6, 8, 12, 16, 24, 36, and 48 hours post-dose; in the MAD part (Part 2), 0.25, 0.5, 0.75, 1, 1.25, 1.5, 1.75, 2, 2.5, 3, 4, 5, 6, 8, hours after the Day 1 dose, 0.25, 0.5, 0.75, 1, 1.25, 1.5, 1.75, 2, 2.5, 3, 4, 5, 6, 8, 12, 16, 24, 36, and 48 hours after the Day 10 dose, and pre-dose on Days 1-5 and 10. Serial blood samples were collected at the following timepoints in VNRX-7145-102: in Part 1 (dosing on Days 1, 5, and 9), predose and 0.5, 0.75, 1, 1.25, 1.5, 1.75, 2, 2.5, 3, 4, 5, 6, 8, 12, 16, 24, and 48 hours postdose; in Part 2, predose on Days 1-5 and Day 10, and 0.5, 0.75, 1, 1.25, 1.5, 1.75, 2, 2.5, 3, 4, 5, 6, and 8 hours after the first dose on Day 1 and the Day 10 dose, and 12, 16, 24, and 48 hours after the Day 10 dose; in Part 3, predose and 0.5, 0.75, 1, 1.25, 1.5, 1.75, 2, 2.5, 3, 4, 5, 6, and 8 hours after the Day 1 and 10 dose and 12, 16, 24, and 48 hours after the Day 10 dose.

In VNRX-7145-101, urine samples for pharmacokinetic analysis were collected predose and at 0 to ≤4, >4 to ≤8, >8 to ≤12, >12 to ≤24, and >24 to ≤48 hours after single doses and on Day 10 following multiple doses. Urine samples were collected in Parts 2 and 3 of VNRX-7145-102 at the following times: predose on Days 1 and 10 and 0 to 4, >4 to ≤8, >8 to ≤12, >12 to ≤24, and >24 to ≤48 hours after the Day 10 dose.

Plasma and urine pharmacokinetics samples were assayed for LED-E, LED, and CTB, as appropriate using validated bioanalytical methods. Liquid chromatography-tandem mass spectrometry (LC-MS/MS) was used to analyze human acidified sodium fluoride potassium oxalate plasma samples for LED-E and LED using a calibration standard of 0.500 to 500 ng/mL (LED-E) and 10.0 to 10,000 ng/mL (LED) based on the analysis of 0.100 mL of acidified plasma. Quantitation used separate weighted 1/x² linear least-squares regression analyses generated from calibration standards. Exploratory screening for metabolites was performed for some samples.

### Pharmacokinetic and Statistical Analyses

For VNRX-7145-101, individual concentration-time data were summarized by cohort using descriptive statistics for all participants who received at least 1 dose of study drug and had at least one evaluable plasma or urine concentration measurement. Single-dose plasma pharmacokinetic parameters were determined in Part 1, and both single-dose (Day 1) and multiple-dose (Day 12) parameters were determined in Part 2. Actual sample times were used to calculate the pharmacokinetic parameters. Calculation of l_{Z} was based on best fit of concentrations in the observed terminal elimination phase of a profile, and statistical inclusion of any parameter based on l_{Z} required the adjusted goodness-of-fit statistic (R²_{adj}) to be 0.8 or higher. Dose proportionality was analyzed using a power model in which pharmacokinetic exposure parameters and dose were converted to the natural log and linear regression results were reported, with the slope as the power estimate. If the 90% CI for the slope contained 1, then the hypothesis of dose proportionality for AUC or Cₘₐₓ could not be rejected. In Part 2, achievement of steady-state concentrations was assessed using Tukey's multiple comparison test for pre-dose and all dosing days (Days 2 to 10) for each dose level separately.

For VNRX-7145-102, descriptive statistics were used to summarize plasma concentrations by study part and treatment at each assessment time point for all participants who received at least 1 dose of study drug and had at least 1 evaluable post-dose concentration measurement. Plasma pharmacokinetic parameters were calculated using noncompartmental analysis of the plasma and urine concentration-time data. Actual sample times were used to calculate the pharmacokinetic parameters. In Part 1, the effect of coadministration of CTB+LED-E on LED-E, LED, cis-CTB, and trans-CTB plasma pharmacokinetic parameters was assessed. AUC_{inf} and Cₘₐₓ were natural log-transformed before analysis, using a linear mixed effects model. The analysis of variance (ANOVA) model included fixed effects for treatment, period, and sequence and a random effect for participant within sequence. Point estimates, geometric means, geometric mean ratios, and corresponding 90% CIs were calculated. An absence of interaction was indicated by a 90% CI that was contained within 0.80 to 1.25. In Parts 2 and 3, plasma and/or urine pharmacokinetic parameters were assessed on Days 1, 2, 3, 4, 5, and 10 (Part 2) and Days 1 and 10 (Part 3).

In both studies, plasma pharmacokinetic parameters were calculated using WinNonlin™ (Certara; Radnor, PA) and urine pharmacokinetic parameters were calculated using SAS™ (SAS Institute; Cary, NC).

Safety analyses included all participants in both VNRX-7145-101 and VNRX-7145-102 who received at least 1 dose of study medication. Plasma and urine concentration and pharmacokinetic parameters were analyzed for all participants who received at least 1 dose of study drug and had at least 1 evaluable post-dose plasma concentration. All safety and pharmacokinetic data were summarized using descriptive statistics including change from baseline, where applicable. Drug-drug interactions between CTB and LED-E were analyzed using an analysis of variance model that included fixed effects for treatment, period, and sequence and a random effect for participant within sequence. An absence of interaction was indicated by a 90% CI contained within an equivalence margin of 0.80 to 1.25.

TABLE 2
Plasma pharmacokinetic parameters for ledaborbactam etzadroxil (LED-E) and
ledaborbactam (LED), following single doses of LED-E)

| | Study VNRX-7145-101^{b} | | | | | |
|---|---|---|---|---|---|---|
| | | | | | | LED-E |
| | LED-E | LED-E | LED-E | LED-E | LED-E | 1000 |
| | 100 mg | 200 mg | 300 mg | 500 mg | 800 mg | mg |
| Parameter^{a} | (n = 6) | (n = 6) | (n = 6) | (n = 6) | (n = 6) | (n = 6) |

LED-E

| | | | | | | |
|---|---|---|---|---|---|---|
| Tₘₐₓ, h^{c} | 0.50 | 0.50 | 0.50 | 0.51 | 0.50 | 0.75 |
| | (0.50- | (0.50- | (0.50- | (0.50- | (0.50- | (0.50- |
| | 1.00) | 1.00) | 1.50) | 2.50) | 1.50) | 0.75) |
| Cₘₐₓ, | 167 | 167 | 347 | 917 | 1160 | 1840 |
| ng/mL | (79.7) | (57.6) | (95.5) | (112) | (95.3) | (21.2) |
| AUC_{inf}, | 177 | 249 | 439 | 1290 | 1720 | 2300 |
| h · ng/mL | (53.4) | (23.3) | (52.7) | (67.4) | (68.0) | (11.2) |
| t_{1/2}, h | 2.81 | 3.34 | 3.63 | 4.70 | 3.37 | 4.59 |
| | (24.5) | (6.35) | (17.3) | (13.3) | (14.2) | (7.73) |
| CL/F, | 564 | 803 | 683 | 389 | 465 | 435 |
| L/h | (53.4) | (23.3) | (52.7) | (67.4) | (68.0) | (11.2) |
| V_{z}/F, L | 2290 | 3870 | 3580 | 2640 | 2260 | 2880 |
| | (50.7) | (22.2) | (62.9) | (62.6) | (68.7) | (15.1) |

LED

| | | | | | | |
|---|---|---|---|---|---|---|
| Tₘₐₓ, h^{c} | 1.25 | 1.50 | 1.25 | 1.26 | 2.00 | 2.00 |
| | (0.50- | (1.00- | (0.50- | (1.00- | (1.00- | (1.00- |
| | 2.00) | 3.00) | 2.00) | 3.00) | 3.00) | 3.00) |
| Cₘₐₓ, | 4010 | 5970 | 7570 | 16,800 | 24,000 | 26,500 |
| ng/mL | (25.6) | (11.8) | (29.3) | (8.43) | (14.2) | (7.92) |
| AUC_{inf}, | 15,400 | 26,200 | 33,100 | 69,300 | 125,000 | 114,000 |
| h · ng/mL | (30.5) | (16.7) | (25.6) | (15.2) | (22.6) | (8.82) |
| t_{1/2}, h | 5.53 | 6.10 | 7.48 | 11.3 | 8.59 | 11.3 |
| | (18.7) | (27.4) | (32.4) | (34.5) | (42.9) | (19.1) |

Except where indicated, data are geometric mean (coefficient of variation, %).
^{a}AUC_{inf}, area under the concentration-time curve through infinity; CI, confidence interval; CL/F, apparent total clearance; Cₘₐₓ, maximum concentration; Tₘₐₓ, time of maximum concentration; V_{z}/F, volume of distribution in the terminal phase.
^{b}LED-E, ledaborbactam etzadroxil.
^{c}Data are median (range).

TABLE 3
Plasma pharmacokinetic parameters for ledaborbactam etzadroxil (LED-E) and
ledaborbactam (LED), on dosing Day 10 following multiple doses of LED-E with or without
ceftibuten (CTB).

| | | | | | | |
|---|---|---|---|---|---|---|
| | | | | | LED-E | LED-E |
| | | LED-E | LED-E | LED-E | 300 mg + | 500 mg + |
| | LED-E 75 | 150 mg | 300 mg | 500 mg | CTB 400 | CTB 400 |
| | mg q8h^{b} | q8h^{b} | q8h^{b} | q8h^{b} | mg q8h^{b} | mg q8h^{b} |
| Parameter^{a} | (n = 9) | (n = 8) | (n = 8) | (n = 9) | (n = 10) | (n = 10) |

LED-E

| | | | | | | |
|---|---|---|---|---|---|---|
| Tₘₐₓ, h^{c} | 0.50 (0.50- | 0.50 (0.25- | 0.50 (0.25- | 0.75 (0.50- | 0.50 (0.50- | 0.51 (0.50- |
| | 0.75) | 0.75) | 0.75) | 1.50) | 1.75) | 1.00) |
| Cₘₐₓ, | 122 (121) | 447 (100) | 629 (103) | 919 (51.6) | 467 (56.5) | 1175 |
| ng/mL | | | | | | (60.4) |
| AUCₜₐᵤ, | 85.5 (88.1) | 309 (72.9) | 480 (61.7) | 1048 | 463 (34.1) | 1090 |
| h · ng/mL | | | | (37.0) | | (28.7) |
| t_{1/2}, h | 3.07 (23.8) | 4.78 (23.4) | 6.75 (18.5) | 9.14 (28.1) | 7.68 (29.6) | 10.7 (23.4) |
| CL_{SS}/F, | 877 (88.1) | 486 (72.9) | 625 (61.7) | 477 (37.0) | — | — |
| L/h | | | | | | |
| V_{z}/F, L | 3880 | 3350 | 6080 | 6289 | — | — |
| | (70.7) | (54.1) | (65.0) | (31.0) | | |

LED

| | | | | | | |
|---|---|---|---|---|---|---|
| Tₘₐₓ, h^{c} | 1.00 (0.50- | 0.75 (0.75- | 1.13 (0.75- | 1.50 (0.75- | 1.00 (0.75- | 1.50 (0.50- |
| | 1.75) | 1.75) | 1.75) | 3.00) | 2.00) | 3.00) |
| Cₘₐₓ, | 3690 | 5870 | 11,600 | 15,788 | 8469 | 16,152 |
| ng/mL | (25.5) | (11.4) | (31.9) | (29.9) | (35.3) | (25.0) |
| AUCₜₐᵤ, | 13,900 | 20,800 | 40,900 | 69,988 | 33,319 | 62,239 |
| h · ng/mL | (16.8) | (15.6) | (13.7) | (26.4) | (25.3) | (16.8) |
| t_{1/2}, h | 9.3 (8.5) | 11.4 (25.0) | 11.3 (10.4) | 12.5 (6.5) | 11.4 (10.6) | 12.3 (8.4) |

Except where indicated, data are geometric mean (coefficient of variation, %).
^{a}AUCₜₐᵤ, area under the concentration-time curve through the last quantifiable concentration; CL_{SS}/F, apparent total clearance at steady state after oral administration; Cₘₐₓ, maximum concentration; CTB, ceftibuten; LED, ledaborbactam; LED-E, ledaborbactam etzadroxil; Tₘₐₓ, time of maximum concentration; V_{z}/F, volume of distribution in the terminal phase.
^{b}q8h, every 8 hours.
^{c}Data are median (range).

TABLE 4
Summary of statistical analysis of potential drug-drug interactions following a single
dose of ledaborbactam etzadroxil (LED-E) 500 mg plus ceftibuten (CTB) 400 mg

| | Pharmacokinetic | | | Ratio of Test/Reference | | |
|---|---|---|---|---|---|---|
| | Parameter | Geometric LS means^{b} | | | 90% CI^{e} | |
| Analyte^{a} | (Unit^{c,d}) | Test | Reference | Estimate | Lower | Upper |
| LED-E^{f} | Cₘₐₓ, μg/mL | 787 | 1037 | 0.76 | 0.66 | 0.87 |
| | AUC_{inf}, | 1068 | 1138 | 0.94 | 0.85 | 1.04 |
| | h · μg/mL | | | | | |
| LED^{f} | Cₘₐₓ, μg/mL | 13,162 | 13,737 | 0.96 | 0.89 | 1.03 |
| | AUC_{inf}, | 65,362 | 66,302 | 0.99 | 0.95 | 1.03 |
| | h · μg/mL | | | | | |
| Cis-CTB^{g} | Cₘₐₓ, μg/mL | 15,405 | 17,562 | 0.88 | 0.79 | 0.97 |
| | AUC_{inf}, | 79,027 | 90,092 | 0.88 | 0.81 | 0.95 |
| | h · μg/mL | | | | | |
| Trans-CTB^{g} | Cₘₐₓ, μg/mL | 1319 | 1552 | 0.85 | 0.77 | 0.94 |
| | AUC_{inf}, | 8946 | 10,404 | 0.86 | 0.79 | 0.93 |
| | h · μg/mL | | | | | |

^{a}Cis-CTB, cis-ceftibuten; LED, ledaborbactam; LED-E, ledaborbactam etzadroxil; trans-CTB, trans-ceftibuten.
^{b}LS, least-squares.
^{c}Units are for geometric LS means, and do not apply to the ratios or corresponding 90% CIs.
^{d}AUC_{inf}, area under the concentration-time curve from time zero to infinity; Cₘₐₓ, maximum concentration.
^{e}CI, confidence interval.
^{f}For LED-E and LED, test product was a single dose of LED-E 500 mg + CTB 400 mg; reference product was a single dose of LED-E 500 mg.
^{g}For CTB, test product was a single dose of LED-E 500 mg + CTB 400 mg; reference product was CTB 400 mg.

### Example 6: Safety and PK of Ceftibuten-LED-E Fixed-Dose Combination (600 mg/600 mg or 1200/1200 mg)

This study is being conducted to assess the pharmacokinetics (PK) of a ceftibuten-LED-E fixed-dose combination (FDC) formulation (administered as ceftibuten-Compound A) and the effects of esomeprazole on the disposition of ceftibuten and ledaborbactam (Compound 2). In addition, this study will evaluate the safety and tolerability of the FDC formulation following multiple-dose administration under fed or fasted conditions.

TABLE 5

| Objectives | Endpoints |
|---|---|
| Primary, Part 1 | |
| To compare rate and extent of plasma exposure | Primary PK endpoints: |
| of ceftibuten, ledaborbactam, and | Area under the concentration-time curve from |
| ledaborbactam etzadroxil when LED-E is | time zero to infinity (AUC0-∞) and maximum |
| administered as an FDC formulation versus co- | observed concentration (Cₘₐₓ) |
| administered as separate capsules | |
| To evaluate the effect of esomeprazole, a proton | Primary PK endpoints: |
| pump inhibitor (PPI), on the plasma PK of | AUC0-∞ and Cₘₐₓ |
| ceftibuten and ledaborbactam | |
| Secondary, Part 1 | |
| To evaluate the safety and tolerability of | Participants experiencing: |
| ceftibuten-LED-E when given alone or with | Treatment-emergent adverse events (TEAEs) |
| esomeprazole | Serious TEAEs |
| | TEAEs leading to discontinuation |
| Primary, Part 2 | |
| To evaluate the safety and tolerability of | Participants experiencing: |
| ceftibuten-LED-E FDC following multiple dose | Treatment-emergent adverse events (TEAEs) |
| administration of ceftibuten-LED-E FDC | Serious TEAEs |
| formulation | TEAEs leading to discontinuation. |
| To evaluate the steady-state plasma and urine | Plasma and urine PK endpoints: |
| PK of ceftibuten and ledaborbactam following | AUC0-τ and Cₘₐₓ |
| multiple dose administration of ceftibuten-LED- | Ae, Fe, and CLR as appropriate |
| E FDC formulation | |

### Overall Design:

The purpose of the study is three-fold:
First, Part 1 of the study will compare the PK of ceftibuten and ledaborbactam etzadroxil when co-administering ceftibuten and LED-E in separate capsules versus when given as FDC capsules (FDC PK).
Second, also in Part 1, the impact of esomeprazole, a PPI, on the PK of ceftibuten and ledaborbactam when given as FDC capsules comprising LED-E and ceftibuten will be evaluated (PPI-DDI).
Third, Part 2 will assess the safety and tolerability of ceftibuten-LED-E following multiple dose administration of ceftibuten-LED-E FDC capsules in fasting or non-fasting conditions.

### Part 1 (FDC PK and PPI DDI)

Part 1 will enroll participants who will receive:
a single oral dose of 600 mg ceftibuten (administered as ceftibuten dihydrate and amount adjusted to be equivalent to 600 mg of ceftibuten) and 600 mg of LED-E (administered as Compound A and amount adjusted to be equivalent to 600 mg of LED-E Compound 1) following a 10-hour fast on 3 occasions in a fixed sequence:

### FDC PK:

Co-administered as separate capsules on Day 1
Administered as FDC capsules on Day 4

### PPI-DDI:

Participants will receive a 40 mg dose of esomeprazole orally once daily in the morning for 5 days (Days 7-11) and a single oral dose of the FDC of 600 mg ceftibuten and 600 mg LED-E on Day 11.

Standardized meals will be provided to participants on days when PK collection occurs (Days 1, 4, and 11).

Blood and urine samples will be collected following each dose of ceftibuten-LED-E to determine plasma and urine concentrations and PK of ceftibuten, ledaborbactam etzadroxil (as appropriate), and ledaborbactam. Sample collection timepoints and intervals are specified in the Schedule of Activities.

### Part 2 (Safety and Tolerability)

Part 2 will enroll participants in two or three groups. Groups 1 and 2 will each receive oral doses of ceftibuten-LED-E, 600 mg of each drug as FDC capsules, every 12 hours over a 7-day treatment period, with a single morning dose on Day 7 (a total of 13 doses). Group 1 will receive their doses after a fasting period, while Group 2 will receive theirs after a meal.

Following a review of data from Groups 1 and 2, a determination will be made regarding whether Group 3 is needed. The number of bowel movements and consistency will be collected and assessed by the CRU staff and recorded from CRU admission through the Follow-up period. Participants will maintain a bowel movement diary during the Follow-up period. Fecal consistency will be captured as hard (stools that are hard, larger or lumpy and/or defecation straining), normal (formed stools, smooth and/or soft and easy to pass), or loose (stools that are mushy or liquid or a mixture of both).

Standardized meals will be provided to participants on Day 7 when PK collection occurs. For each group, blood and urine samples will be collected to determine plasma and urine concentrations and steady-state PK of ceftibuten and ledaborbactam.

### Intervention Groups and Duration:

### Part 1:

All study interventions will be administered following a 10-hour fast before and 4-hour fast after dosing. Detailed dosing regimen is as follows:
Period 1, Day 1: Ceftibuten 600 mg+LED-E 600 mg administered as a single oral dose as three 200 mg ceftibuten capsules and three 200 mg LED-E capsules (administered as Compound A).
Period 2, Day 4: Ceftibuten-LED-E FDC 600 mg/600 mg administered as a single oral dose of 3 capsules each containing 200 mg of ceftibuten and 200 mg of LED-E (administered as Compound A).
Period 3, Days 7-10: Esomeprazole 40 mg administered as a single oral dose in the morning. Fasting is not required on days when esomeprazole is given alone.
Period 3, Day 11: Ceftibuten-LED-E FDC 600 mg/600 mg administered as a single oral dose of 3 capsules each containing 200 mg of ceftibuten and 200 mg of LED-E (administered as Compound A), administered with a single oral dose of esomeprazole 40 mg in the morning.

### Part 2:

Oral doses of ceftibuten-LED-E administered as FDC capsules every 12 hours over a 7-day treatment period, with a single morning dose on Day 7 (total of 13 doses). Detailed dosing regimen is as follows:

### Days 1-6

Group 1: 600 mg-600 mg ceftibuten-LED-E with a 2-hour fast before and a 2-hour fast after dosing. Participants who experience gastrointestinal symptoms (e.g., frequent and/or unformed stools) will be permitted to have a meal 30 minutes prior to dose administration, at the investigator's discretion.
Group 2: Following a 2-hour fast, participants will receive a meal. Dosing of 600 mg-600 mg ceftibuten-LED-E will occur 30 minutes after the start of the meal.

### Day 7

Group 1: 600 mg-600 mg ceftibuten-LED-E with a 10-hour fast before and a 4-hour fast after dosing. A standardized meal will be provided for all meals. Participants who experience gastrointestinal symptoms (e.g., frequent and/or unformed stools) will be permitted to have a meal 30 minutes prior to dose administration, at the investigator's discretion.
Group 2: Following a 10-hour fast, participants will receive a standardized meal. Dosing with 600 mg-600 mg ceftibuten-LED-E will occur 30 minutes after the start of the standardized meal.

### Example 7: Ceftibuten-LED-E in Complicated Urinary Tract Infection

This study would be conducted to evaluate the efficacy and pharmacokinetics/pharmacodynamics of a ceftibuten-LED-E fixed-dose combination (FDC) formulation (administered as ceftibuten-Compound A) for the treatment of complicated urinary tract infection (cUTI). Two cohorts will be recruited to evaluate three different dosing regimens:
Ceftibuten-LED-E FDC 600 mg/600 mg administered as a single oral dose of 3 capsules each containing 200 mg of ceftibuten and 200 mg of LED-E (administered as Compound A) every 12 hours (q12h).
Ceftibuten-LED-E FDC 400 mg/400 mg administered as a single oral dose of 2 capsules each containing 200 mg of ceftibuten and 200 mg of LED-E (administered as Compound A) every 8 hours (q8h).

### Example 8: Assessment of the In Vivo Efficacy of Ceftibuten-Ledaborbactam Combination Against Serine-β-Lactamase-Producing Enterobacterales in a Neutropenic Murine Complicated Urinary Tract Infection Model

The aim of this study was to characterize the antibacterial activity of humanized exposures of ceftibuten in combination with various ledaborbactam humanized exposures against β-lactamase-producing Enterobacterales over a 24-hour (h) treatment period using a previously published and validated murine complicated UTI (cUTI) model (Monogue et al., Antimicrobial Agents and Chemotherapy 62(9):e02596-17 (2018); Clinical and Laboratory Standards Institute. Performance Standards for Antimicrobial Susceptibility Testing: Approved. 34th ed. CLSI; 2024).

### Materials and Methods

### Antimicrobial Agents and Doses

For the in vivo studies, ledaborbactam (Compound 2, concentration: 1 mg/mL) and ceftibuten (concentration: 2 mg/mL) were reconstituted with sodium phosphate buffer (50 mM, pH 8). Subsequent ledaborbactam and ceftibuten dilutions in buffer were made to attain final concentrations that would deliver the required amounts based on the mean weight of the study mice population. Both ledaborbactam and ceftibuten were administered via subcutaneous (SC) injections of 0.1 mL.

The doses of the humanized ceftibuten regimens in combination with ledaborbactam (i.e., 400/400 mg q8 h and 600/600 mg q12h) utilized in the in vivo efficacy studies were determined via pharmacokinetic studies and are shown in Table 6.

### Animal Infection Model

### Animals

Female, specific-pathogen-free, CD-1 mice (weight: 20-22 g) were obtained from Charles River Laboratories, Inc. (Raleigh, North Carolina, USA). All animals were allowed to acclimatize for 48 hours before experimentation. During acclimation, animals were housed in groups of six mice at controlled room temperature in High-Efficiency Particulate Air (HEPA)-filtered cages (Innovive, San Diego, CA, USA). Cages were supplemented with nesting material for enrichment purposes. Study rooms were maintained with diurnal cycles (12 h light/12 h dark). Food and water were provided ad libitum. Monitoring was conducted at least three times per day for signs of morbidity. Neutropenic Complicated Urinary-tract Infection Model

The neutropenic murine direct kidney inoculation model was conducted as previously described (Monogue et al.; CLSI as cited above). Briefly, mice were rendered neutropenic after receiving 150 and 100 mg/kg cyclophosphamide intraperitoneal (IP) injections 4 and 1 days prior to inoculation, respectively. Uranyl nitrate administered as a 5 mg/kg IP injection on day −3 was used to create a predictable degree of renal impairment. The area over only the left kidney (flank) was shaved and prepped for surgical incision. Mice were anesthetized during the inoculation period with ketamine/xylazine/acepromazine 100/10/3 mg/kg administered as an IP injection. Buprenorphine 0.05 mg/kg IP was administered prior to inoculation/surgical incision and 6 hours post-surgery to alleviate pain/distress.

Prior to incision, a bacterial suspension (108 colony forming units (CFU)/mL) was prepared and diluted (1:20) for kidney inoculation. The predicted incision site was sterilized with a Chloraprep® (Care Infusion; El Paso, TX, USA) scrub-brush and povidine-iodine solution (Medline Inc.; Northfield, IL, USA). Mice were held in lateral position and a small superficial incision was placed in the flank region to remove the top skin layer which allowed for left kidney visualization; subsequently, 0.05 mL of diluted bacterial suspension was injected directly into the kidney. The skin incision was then closed using 4.0 Vicryl suture with RB-1 material (Ethicon Inc; Somerville, NJ, USA). Local anesthetic bupivacaine (0.1% working solution) was administered (infiltrating the incision line) immediately post-incision. Mice were then randomized into respective groups. Three hours were allotted prior to dosing and/or harvesting interventions to allow for establishment of infection for all in vivo assessments. All pharmacokinetic and pharmacodynamic time-point determinations were relative to start of treatment or intervention (0h) rather than time of inoculation. If mice were found moribund after treatment intervention, they were compassionately euthanized, and tissues were harvested.

### In Vivo Efficacy Studies

The purpose of these studies was to assess the in vivo efficacy of various humanized exposures of ceftibuten/ledaborbactam administered with either an 8 hour or 12-hour dosing frequency against β-lactamase-producing Enterobacterales isolates over 24 h in the neutropenic murine cUTI infection model.

One group of 6 mice was sacrificed at 0 h and their infected left kidneys (n=6) and bladders (n=6) were aseptically harvested as described above to serve as 0 h control. The remaining groups (6 mice per group) were exposed to one of the following interventions: 24 h saline control, ceftibuten 400 mg q8h, ceftibuten 600 mg q12h, ceftibuten/ledaborbactam 400/100 mg q8h, ceftibuten/ledaborbactam 400/200 mg q8h, ceftibuten/ledaborbactam 400/400 mg q8h, ceftibuten/ledaborbactam 600/150 mg q12h, ceftibuten/ledaborbactam 600/300 mg q12h, and ceftibuten/ledaborbactam 600/600 mg q12h. At 24h, each mouse kidney and bladder were aseptically harvested, plated, and enumerated as described above. Multiple unpaired t-tests (with Welch correction) were used to compare efficacy between the ceftibuten/ledaborbactam 400/400 mg q8h regimen and ceftibuten/ledaborbactam 600/600 mg q12 h regimen to assess the impact of dosing frequency for each isolate tested. A P value ≤0.05 was defined as statistically significant.

### Results

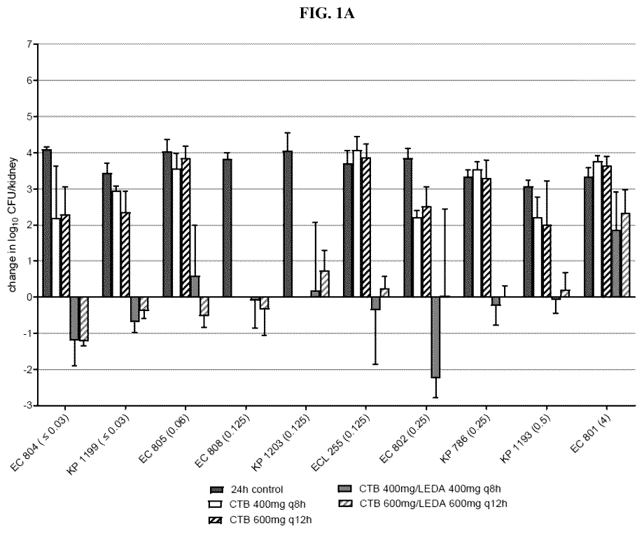

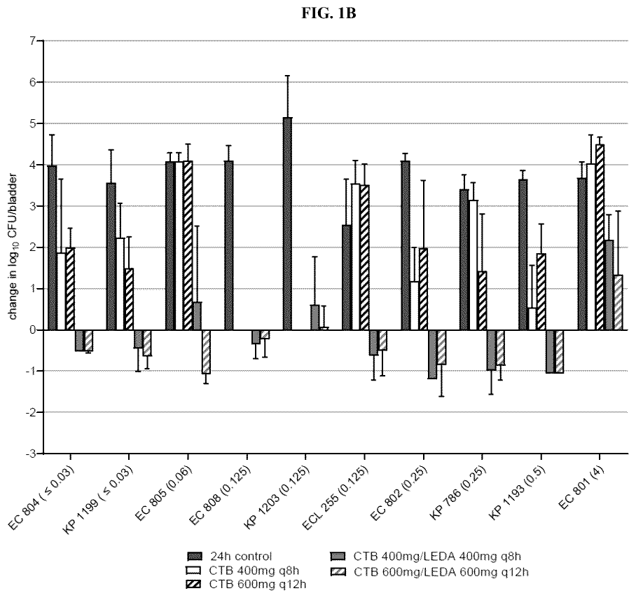

Ten isolates were evaluated in the in vivo efficacy studies. These 10 isolates were chosen due to specific target genus/species, genotypes, and ceftibuten/ledaborbactam MICs (indicated in Tables 7 and 8). Summary graphs of in vivo efficacy displaying change in bacterial density from 0h controls as well as absolute bacterial density in the kidney and bladder are presented in FIG. 1A and FIG. 1B.

In the kidney infection model, 0h control mice displayed an overall mean log₁₀ CFU/kidney of 5.54±0.47 across all isolates examined, which increased to 9.23±0.33 log₁₀ CFU/kidney in untreated (saline-dosed) mice after 24 hours. Relative to 0h control, both ceftibuten 400 mg q8 h and ceftibuten 600 mg q12 h monotherapy resulted in mean bacterial growth of 3.08±0.93 and 3.22±0.97 log₁₀ CFU/kidney at 24 hours, respectively.

Among the 9 out of 10 Enterobacterales with ceftibuten/ledaborbactam MIC ≤0.5 mg/L, treatment with ceftibuten/ledaborbactam 400/400 mg q8 h resulted in a change of bacterial density ranging from 0.59 to −2.25 (median −0.24, inter quartile range (IQR) −0.68 to −0.08) log₁₀ CFU/kidney, relative to 0h. Against these isolates, ceftibuten/ledaborbactam 600/600 mg q12 h also resulted in a change of bacterial density ranging from 0.74 to −1.22 (median 0.03, IQR −0.38 to 0.21) log₁₀ CFU/kidney. Bacterial growth of >1-log was observed in the isolate (EC 801) with an elevated ceftibuten/ledaborbactam MIC of 4 mg/L after treatment with both ceftibuten/ledaborbactam 400/400 mg q8 h and ceftibuten/ledaborbactam 600/600 mg q12h.

The administration of ceftibuten/ledaborbactam 400/100 mg q8 h and 600/150 mg q12h resulted in bacterial density changes ranging from 0.70 to −1.77 log₁₀ (median −0.09, IQR −1.02 to 0.24) CFU/kidney and 1.42 to −1.83 (median 0.67, IQR −0.20 to 0.87) log₁₀ CFU/kidney respectively, among 7 Enterobacterales with ceftibuten/ledaborbactam MIC ≤0.5 mg/L. Similarly, administration of ceftibuten/ledaborbactam 400/200 mg q8 h and 600/300 mg q12 h resulted in bacterial density changes ranging from 0.42 to −0.41 log₁₀ CFU/kidney (median −0.19, IQR −0.26 to −0.06) and 1.05 to −0.82 (median 0.41, IQR −0.48 to 0.62) log₁₀ CFU/kidney, respectively against these isolates.

In vivo efficacy results in the bladder mirrored the trends from their kidney counterparts. Average bacterial density was 2.95±0.62 at 0 hour and 6.77±0.76 log₁₀ CFU/bladder in saline-dosed control groups after 24 hours. In mice receiving humanized ceftibuten alone, the 400 mg q8 h and 600 mg q12 h regimens resulted in average net growth of 2.56±1.58 and 2.75±1.41 log₁₀ CFU/bladder, respectively.

Among the 9 out of 10 Enterobacterales with ceftibuten/ledaborbactam MIC ≤0.5 mg/L, the administration of ceftibuten/ledaborbactam 400/400 mg q8 h and ceftibuten/ledaborbactam 600/600 mg q12 h was associated with bacterial density changes ranging from 0.68 to −1.19 (median −0.53, IQR −0.99 to −0.39) log₁₀ CFU/bladder and 0.08 to −1.08 (median −0.65, IQR −0.86 to −0.50) log₁₀ CFU/bladder compared with starting inoculum, respectively. Notably, bacterial density reductions reached the lower limit of detection across several isolates due to the lower starting inoculum and larger magnitude of ceftibuten/ledaborbactam activity in the bladder. In all strains except EC 805, activity of ceftibuten/ledaborbactam 400/100 mg q8h, 600/150 mg q12 h and ceftibuten/ledaborbactam 400/200 mg q8h, 600/300 mg q12 h were comparable to the activity of ceftibuten/ledaborbactam 400/400 mg q8 h and ceftibuten/ledaborbactam 600/600 mg q12h. The observed log₁₀ CFU/bladder counts from efficacy studies are listed in Appendix C.

There was no statistical difference (p>0.05) between the two high dose regimens ceftibuten/ledaborbactam 400/400 mg q8 and 600/600 mg q12 h when tested against each isolate included in the in vivo efficacy studies (Table 7 and Table 8).

### CONCLUSION

The three potential clinical exposures of ceftibuten/ledaborbactam administered every 8 hours or 12 hours resulted in bacteriostasis to 1-log bacterial reduction against a variety of ceftibuten-resistant Enterobacterales bacteria harboring a broad range of enzyme-mediated resistance mechanisms (ESBL, KPC, and OXA-48) in the neutropenic murine cUTI infection model. These results also corroborate the in vivo activity of ceftibuten/ledaborbactam previously observed in the murine thigh infection model (Fratoni et al., Journal of Antimicrobial Chemotherapy 78(1):93-100 (2022)). No statistically significant difference in efficacy was observed between the two highest dosing regimens (ceftibuten/ledaborbactam 400/400 mg q8 h and 600/600 mg q12h) despite varying the administration frequency i.e., q8 h vs q12h.

TABLE 6
Doses used in the study

| | Human dose | Murine dose |
|---|---|---|
| Ceftibuten | 400 mg q8h | 0 h 3 mg/kg; MD: 2.25 mg/kg q8h |
| | 600 mg q12h | 0 h 4.25 mg/kg; MD: 3.18 mg/kg |
| | | q12h |
| Ledaborbactam | 100 mg q8h | 0 h 0.2 mg/kg; MD: 0.12 mg/kg q8h |
| | 200 mg q8h | 0 h 0.4 mg/kg; MD: 0.24 mg/kg q8h |
| | 400 mg q8h | 0 h 0.8 mg/kg; MD: 0.48 mg/kg q8h |
| | 150 mg q12h | 0 h 0.3 mg/kg; MD: 0.18 mg/kg q12h |
| | 300 mg q12h | 0 h 0.6 mg/kg; MD: 0.36 mg/kg q12h |
| | 600 mg q12h | 0 h 1.2 mg/kg; MD: 0.72 mg/kg q12h |

MD = Maintenance dose

TABLE 7
Results of the multiple unpaired t-test (with Welch correction) between
ceftibuten/ledaborbactam 400/400 mg q8h and ceftibuten/ledaborbactam
600/600 q12h on change in CFU from 0 h in kidney per isolate

| | | Mean absolute log10 | |
|---|---|---|---|
| Isolate No. | Mean absolute log10 | CFU/kidney in CTB | |
| (CTB/LEDA; | CFU/kidney in CTB | 600/LED 600 mg q12h | |
| MIC mg/L)^{a} | 400/LED 400 mg q8h group | group | P-value |
| EC 804 (≤0.03) | −1.2 | −1.22 | 0.9478 |
| KP 1199 (≤ 0.03) | −0.68 | −0.38 | 0.0747 |
| EC 805 (0.06) | 0.59 | −0.53 | 0.1111 |
| EC 808 (0.125) | −0.09 | −0.34 | 0.5716 |
| KP 1203 (0.125) | 0.19 | 0.74 | 0.5184 |
| ECL 255 (0.125) | −0.36 | 0.25 | 0.3725 |
| EC 802 (0.25) | −2.25 | 0.04 | 0.0677 |
| KP 786 (0.25) | −0.24 | 0.03 | 0.3549 |
| KP 1193 (0.5) | −0.08 | 0.21 | 0.2933 |
| EC 801 (4) | 1.86 | 2.33 | 0.3894 |

^{a}MICs determined with ledaborbactam at a fixed concentration of 4 mg/L

TABLE 8
Results of the multiple unpaired t-test (with Welch correction) between
ceftibuten/ledaborbactam 400/400 mg q8h and ceftibuten/ledaborbactam
600/600 q12h on change in CFU from 0 h in bladder per isolate

| | | Mean absolute log10 | |
|---|---|---|---|
| Isolate No. | Mean absolute log10 | CFU/bladder in CTB | |
| (CTB/LED; | CFU/bladder in CTB | 600/LED 600 mg q12h | |
| MIC mg/L)^{a} | 400/LED 400 mg q8h group | group | P-value |
| EC 804 (≤0.03) | −0.53 | −0.52 | 0.6060 |
| KP 1199 (≤0.03) | −0.45 | −0.65 | 0.4610 |
| EC 805 (0.06) | 0.68 | −1.08 | 0.0661 |
| EC 808 (0.125) | −0.34 | −0.23 | 0.6416 |
| KP 1203 (0.125) | 0.61 | 0.08 | 0.3878 |
| ECL 255 (0.125) | −0.62 | −0.5 | 0.7404 |
| EC 802 (0.25) | −1.19 | −0.85 | 0.3794 |
| KP 786 (0.25) | −0.99 | −0.86 | 0.6776 |
| KP 1193 (0.5) | −1.06 | −1.06 | —* |
| EC 801 (4) | 2.19 | 1.33 | 0.2528 |

*No p-value calculated as all absolute CFU results were at lower limit of detection with SD ± 0
^{a}MICs determined with ledaborbactam at a fixed concentration of 4 mg/L

### Example 9: Preparation of Fixed Dose Combination (FDC) Capsule Formulations of Ceftibuten Dihydrate-Compound 1 Citrate Coordination Complex (Compound A)

### General Description of Capsule Manufacturing Process

The process to manufacture solid oral dosage forms of ceftibuten dihydrate/Compound A allows for formulations containing ceftibuten dihydrate and Compound A at any ratio, along with the addition of common excipients to facilitate processing (fillers, binders, lubricants, glidants etc.) or excipients to adjust the pharmaceutical properties of the capsule (disintegrants).

The manufacturing process involves combining the two drug substances and desired excipients and blending the mixture to produce a homogeneous powder. The resulting blend was then densified using roller compaction to produce ribbons, which were broken down into granules by milling through a screen. The roller compaction process was repeated on the granules to further densify the granules and to ensure uniformity of the blend. The final granules were then encapsulated into the appropriate size capsules and sealed using an automated capsule banding machine. The physical and chemical stability of the capsules were evaluated using standard analytical and pharmaceutical techniques.

### Preparation of 200 mg Compound 1/200 mg Ceftibuten Capsules

Fixed dose capsules that contained the equivalent to 200 mg ceftibuten (as ceftibuten dihydrate) and the equivalent to 200 mg of Compound 1 (as Compound A) were prepared from the formulation composition shown in Table 9.

TABLE 9
Formulation of 200 mg Compound 1 and
200 mg Ceftibuten FDC Capsules

| | | | Weight/Capsule |
|---|---|---|---|
| Ingredients | Function | % w/w | (mg) |
| Ceftibuten Dihydrate^{a} | Drug Substance | 39.58 | 229.4 |
| Compound A^{b} | Drug Substance | 50.11 | 290.4 |
| Form A | | | |
| Microcrystalline | Diluent | 8.73 | 50.6 |
| Cellulose | | | |
| Magnesium Stearate | Lubricant | 0.40 | 2.3 |
| Colloidal Silica | Glidant | 0.40 | 2.3 |
| Sodium Starch | Disintegrant | 0.79 | 4.6 |
| Glycolate | | | |

| | | |
|---|---|---|
| Total | 100.00 | 579.6 |

^{a}Equivalent to 200.0 mg of ceftibuten per capsule
^{b}Equivalent to 200.0 mg of Compound 1 per capsule (correction factor × 0.6922)

Each of the ingredients were weighed out, screened to eliminate any lumps, and transferred to a glass jar. The resulting mixture was blended until uniform using a Turbula® mixer and the homogeneous powder blend was transferred to the feed hopper and compressed into ribbons using a Freund Vector TFC-Lab roller compactor. The ribbons were milled through a stainless-steel screen to yield a coarse powder, which was further densified by a second pass through the roller compactor followed by screening to yield the final granules. The granules were encapsulated into Size 00, gelatin capsules using a manual 100-unit capsule tray filler with a target fill weight of 579.6 mg. The capsules were then band-sealed using a Schaeffer Technologies lab scale banding machine.

### Example 10: In Vitro and In Vivo Comparison of Compound A/Ceftibuten Dihydrate FDC Capsules with Co-Dosed Compound B Capsules and Ceftibuten Capsules

### Self-Emulsifying Drug Delivery System (SEDDS) Capsules of Compound B

The 200 mg SEDDS capsules were prepared from a 400 mg/mL Compound 1 (LED-E) solution. A 20 mL batch of 400 mg/mL Compound 1 SEDDS formulation was prepared by combining 8.71 g of Compound B and 12.3 g of the vehicle stock (20/20/60 v % of Propylene glycol/PEG-400/Tocophersolan) into a 100 mL round bottom flask outfitted with a magnetic stir bar and mixing at ~60° C. on a hot plate until a clear, homogeneous solution was obtained. The resulting solution was filled, by volume (0.500 mL) into a Size 1 white opaque HPMC capsule using a positive displacement pipette. The resulting capsules were stored at 2-8° C. prior to use.

### In Vitro Study

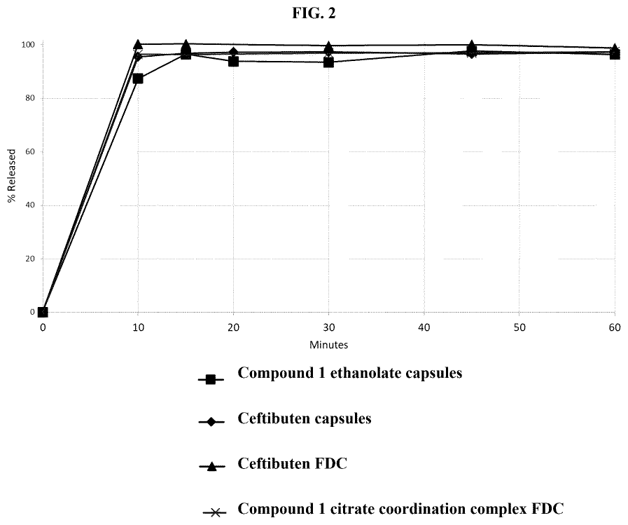

Dissolution testing (Apparatus II method) performed using 900 mL of pH 6.8 50 mM sodium bicarbonate buffer solution and a 75 RPM paddle speed on the FDC capsule prototypes described above showed comparable release of ceftibuten and Compound A to the “co-dosed” (separate capsules) formulations of Compound B and ceftibuten as shown in FIG. 2. Although the SEDDS based capsule formulation of Compound B exhibited immediate release of Compound 1, the formulation was not stable when placed on stability at standard ICH stability conditions of 25° C./60% R.H. and 40° C./65% R.H. Table 10 shows the HPLC purity (Peak Area %) for the Compound B and Compound A capsule formulations.

TABLE 10
Stability Summary of Formulations for Compound
B SEDDS Capsules and Compound A FDC Capsules

| % HPLC Purity of Compound 1 Parent | |
|---|---|
| Compound B SEDDS | Compound A FDC |
| Capsules | Capsules |

| Time (M) | 25° C. | 40° C. | 25° C. | 40° C. |
|---|---|---|---|---|
| 0 | 98.9% | 98.9% | 100.0% | 100.0% |
| 1 | 99.1% | 92.7% | 100.0% | 100.0% |
| 2 | 97.2% | 88.8% | 100.0% | 100.0% |
| 3 | 93.2% | 85.1% | 100.0% | 100.0% |
| 4 | 90.9% | 82.8% | 100.0% | 100.0% |
| 5 | 89.1% | 76.1% | 100.0% | 100.0% |
| 6 | 87.4% | 74.4% | 100.0% | 100.0% |

### In Vivo Study

A single dose comparative PK study with the FDC and the single component formulations was conducted in cynomolgus monkeys. The study was performed by administering either a single FDC capsule (200 mg ceftibuten/200 mg Compound A or two capsules (200 mg ceftibuten and 200 mg Compound B SEDDS formulation). Each formulation was dosed in six monkeys (n=3/sex) with a seven-day wash-out period between the cohorts. Ceftibuten has been reported to have low bioavailability (~20%) in monkeys so ceftibuten PK was not evaluated in this study. Standard pharmacokinetic parameters for Compound 2 (LED) were calculated for both formulations and the average values are reported in Table 11. In addition, the oral bioavailability of Compound 2 from each formulation was estimated based on an existing intravenous PK study in cynomolgus monkeys with Compound 2 (also presented in Table 11). The two formulations showed comparable plasma levels of Compound 2 and Compound 1 but the oral bioavailability of Compound 2 was higher from the formulation containing Compound A.

TABLE 11
Formulation Comparison Pharmacokinetic Parameters in non-Human Primates

| | Co-Dosed Capsules | | | Fixed Dose Combination Capsule | | |
|---|---|---|---|---|---|---|
| | (Compound B SEDDS Capsules | | | (200 mg Ceftibuten/200 mg | | |
| | and Ceftibuten Capsules) | | | Compound A) | | |
| | Male | Female | Average | Male | Female | Average |
| Parameter | (n = 3) | (n = 3) | (n = 6) | (n = 3) | (n = 3) | (n = 6) |
| AUCₗₐₛₜ | 115491 ± | 184236 ± | 149780 ± | 192666 ± | 255975 ± | 224321 ± |
| (hr * ng/mL)^{a} | 66177 | 22397 | 58117 | 64826 | 80087 | 73817 |
| AUCₗₐₛₜ/Dose^{b} | 3602 ± | 3242 ± | 3423 ± | 6216 ± | 4567 ± | 5292 ± |
| ([ng * hr/mL]/ | 1908 | 397 | 1248 | 1657 | 1258 | 1596 |
| [mg/kg]) | | | | | | |
| Cₘₐₓ (ng/mL) | 28567 ± | 40800 ± | 34683 ± | 21870 ± | 43000 ± | 32435 ± |
| | 7343 | 10413 | 10480 | 11437 | 23409 | 20136 |
| Cₘₐₓ/Dose^{b} | 811 ± 215 | 718 ± 181 | 811 ± 215 | 714 ± 386 | 761 ± 383 | 737 ± 345 |
| ([ng/mL]/[mg/kg]) | | | | | | |
| Tₘₐₓ (hr)^{c} | 2 (0.5-2) | 1 (1-4) | 1.5 (0.5-4) | 4 | 4 | 4 |
| Estimated F% (%)^{d} | 42.3 ± 22.4 | 38.1 ± 4.67 | 40.2 ± 14.7 | 73.1 ± 19.5 | 53.7 ± 14.8 | 63.4 ± 18.8 |

NOTE:
The study administered a fixed dose and was not adjusted to account for the animal's weight, resulting in higher group mean PK parameter values for females. There were no gender-related differences in exposure.
^{a}Total area under the plasma concentration-time curve, calculated to the last observable time point,
^{b}AUCₗₐₛₜ and Cₘₐₓ normalized to each animal's weight-adjusted dose of Compound 1 in mg/kg
^{c}Median (min-max). Where no min-max range given in parentheses, Tₘₐₓ occurred at the same time in all animals
^{d}Estimated oral bioavailability (F %) was calculated using each animal's Compound 2 dose-normalized AUCₗₐₛₜ (calculated by dividing each animal's AUCₗₐₛₜ/dose by a correction factor (MW_{Compound 2}/MW_{Compound 1}), and dividing the Compound 2 dose-normalized AUCₗₐₛₜ by the mean AUCₗₐₛₜ value resulting from IV administration of 1 mg/kg of Compound 2 to cynomolgus monkeys (data generated in a previous study).

The FDC capsule made from Compound A results in better overall in vivo exposure and oral bioavailability for Compound 2 compared to the SEDDS based capsules made from Compound B. The average AUC value is ~50% higher and the Tₘₐₓ is less variable than the SEDDS capsule, resulting in an ~60% increase in estimated oral bioavailability of Compound 2.

1. A method of treating a bacterial infection in a subject in need thereof, the method comprising co-administering:
(i) a compound that is 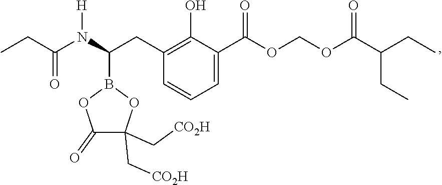 [SMILES: CCC(=O)N[C@@H](Cc1cccc(C(=O)OCOC(=O)C(CC)CC)c1O)B1OC(=O)C(CC(=O)O)(CC(=O)O)O1]
or a pharmaceutically acceptable salt or a solvate thereof; and
(ii) ceftibuten in a daily amount greater than 400 mg.

2. The compound of claim 1, wherein the compound is not a pharmaceutically acceptable salt or a solvate thereof.

3. The method of claim 1, wherein the daily amount of ceftibuten is 600 mg.

4. The method of claim 3, wherein ceftibuten is in the form of ceftibuten dihydrate.

5. The method of claim 4, wherein the daily amount of ceftibuten dihydrate administered is equivalent to 600 mg of ceftibuten.

6. The method of claim 3, wherein ceftibuten is administered every 12 hours (q12h) for a daily amount of 600 mg.

7. The method of claim 3, wherein ceftibuten is administered every 8 hours (q8h) for a daily amount of 600 mg.

8. The method of claim 1, wherein the daily amount of ceftibuten is 800 mg.

9. The method of claim 8, wherein ceftibuten is in the form of ceftibuten dihydrate.

10. The method of claim 9, wherein the daily amount of ceftibuten dihydrate administered is equivalent to 800 mg of ceftibuten.

11. The method of claim 8, wherein ceftibuten is administered every 12 hours (q12h) for a daily amount of 800 mg.

12. The method of claim 8, wherein ceftibuten is administered every 8 hours (q8h) for a daily amount of 800 mg.

13. The method of claim 1, wherein the daily amount of ceftibuten is 1000 mg.

14. The method of claim 13, wherein ceftibuten is in the form of ceftibuten dihydrate.

15. The method of claim 14, wherein the daily amount of ceftibuten dihydrate administered is equivalent to 1000 mg of ceftibuten.

16. The method of claim 13, wherein ceftibuten is administered every 12 hours (q12h) for a daily amount of 1000 mg.

17. The method of claim 13, wherein ceftibuten is administered every 8 hours (q8h) for a daily amount of 1000 mg.

18. The method of claim 1, wherein the daily amount of ceftibuten is 1200 mg.

19. The method of claim 18, wherein ceftibuten is in the form of ceftibuten dihydrate.

20. The method of claim 19, wherein the daily amount of ceftibuten dihydrate administered is equivalent to 1200 mg of ceftibuten.

21. The method of claim 18, wherein ceftibuten is administered every 12 hours (q12h) for a daily amount of 1200 mg.

22. The method of claim 18, wherein ceftibuten is administered every 8 hours (q8h) for a daily amount of 1200 mg.

23. The method of claim 1, wherein the compound is administered in a daily amount that is equivalent to 600 mg of

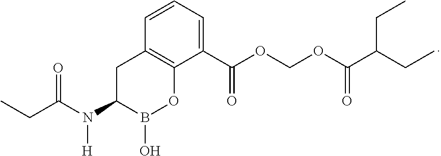 [SMILES: CCC(=O)N[C@H]1Cc2cccc(C(=O)OCOC(=O)C(CC)CC)c2OB1O]

24. The method of claim 1, wherein the compound is administered in a daily amount that is equivalent to 800 mg of

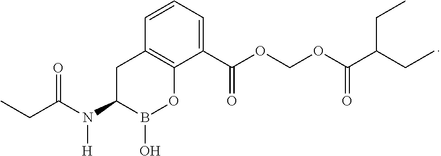 [SMILES: CCC(=O)N[C@H]1Cc2cccc(C(=O)OCOC(=O)C(CC)CC)c2OB1O]

25. The method of claim 1, wherein the compound is administered in a daily amount that is equivalent to 1000 mg of

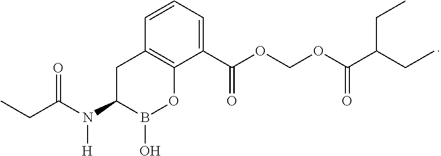 [SMILES: CCC(=O)N[C@H]1Cc2cccc(C(=O)OCOC(=O)C(CC)CC)c2OB1O]

26. The method of claim 1, wherein the compound is administered in a daily amount that is equivalent to 1200 mg of

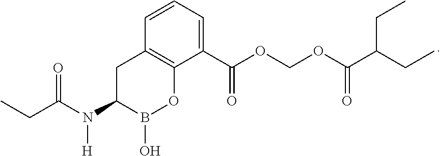 [SMILES: CCC(=O)N[C@H]1Cc2cccc(C(=O)OCOC(=O)C(CC)CC)c2OB1O]

27. The method of claim 1, wherein the compound is administered every 12 hours (q12h).

28. The method of claim 1, wherein the compound is administered every 8 hours (q8h).

29. The method of claim 1, wherein the bacterial infection is caused by carbapenem-resistant Enterobacteriaceae (CRE) or extended-spectrum beta-lactamase (ESBL) producing gram-negative bacteria.

30. The method of claim 1, wherein the bacterial infection is bronchitis, enteritis, gastroenteritis, gonorrhea, an intra-abdominal infection, Lyme disease, otitis media, pharyngitis, pneumonia, a respiratory tract infection, a skin infection, strep throat, tonsillitis, or a urinary tract infection.

31. The method of claim 30, wherein the bronchitis is acute bacterial exacerbations of chronic bronchitis (ABECB).

32. The method of claim 30, wherein the otitis media is acute bacterial otitis media (AOM).

33. The method of claim 30, wherein the respiratory tract infection is an upper respiratory tract infection.

34. The method of claim 30, wherein the respiratory tract infection is a lower respiratory tract infection.

35. The method of claim 1, wherein the bacterial infection is a urinary tract infection.

36. The method of claim 1, wherein the urinary tract infection is complicated urinary tract infection.

37. The method of claim 1, wherein the bacterial infection is caused by Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Borrelia burgdorferi, Vibrio cholerae, Vibrio parahaemolyticus, Legionella pneumophila, Neisseria gonorrhoeae, Neisseria meningitidis, Moraxella, Streptococcus pneumoniae, Streptococcus agalactiae, or Streptococcus pyogenes.

38. The method of claim 1, wherein the bacterial infection is caused by Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Vibrio cholerae, Vibrio parahaemolyticus, Legionella pneumophila, Listeria monocytogenes, Neisseria gonorrhoeae, Neisseria meningitidis, or Moraxella.
